(19) United States
(12) Patent Application Publication
(10) Pub. No.: US 2026/0056205 A1
(43) Pub. Date: Feb. 26, 2026

(54) METHOD TO DISTINGUISH ASPARTATE FROM ISOASPARTATE IN A PROTEIN

(71) Applicant: Wisconsin Alumni Research Foundation, Madison, WI (US)

(72) Inventors: Joshua Coon, Middleton, WI (US); Marcel Morgenstern, Madison, WI (US); Alexander Hebert, Belleville, WI (US)

(73) Assignee: Wisconsin Alumni Research Foundation, Madison, WI (US)

(21) Appl. No.: 19/306,849

(22) Filed: Aug. 21, 2025

Related U.S. Application Data
(60) Provisional application No. 63/686,026, filed on Aug. 22, 2024.

(51) Int. Cl. G01N 33/68; G01N 33/58

(52) U.S. Cl. CPC G01N 33/6848; G01N 33/58; G01N 2333/91017; G01N 2458/15

(57) ABSTRACT

Methods and compositions for detecting isoaspartate residues in polypeptides using mass spectrometry, and for distinguishing isoaspartate residues from aspartate and/or asparagine residues in a polypeptide using MS analysis. Polypeptides containing one or more isoaspartate residues, or suspected of containing one or more isoaspartate residues, are treated with L-isoaspartyl methyltransferase (PIMT), which is able to selectively modify isoaspartate and impart a mass change to the amino acid residue. The polypeptide is then analysed using mass spectrometry, where a mass change caused by the PIMT reaction is detected and characterized. Preferably, these methods are able to be performed without the use of liquid chromatography or other similar separation techniques prior to mass spectrometry analysis.

### CROSS-REFERENCE TO RELATED APPLICATIONS

This application claims the benefit of priority to U.S. Provisional Patent Application No. 63/686,026 filed Aug. 22, 2024, which is hereby incorporated by reference in its entirety to the extent not inconsistent herein.

### STATEMENT REGARDING FEDERALLY SPONSORED RESEARCH

This invention was made with government support under GM108538 awarded by the National Institutes of Health. The government has certain rights in the invention.

### REFERENCE TO AN ELECTRONIC SEQUENCE LISTING

The contents of the electronic sequence listing (42-24 seq listing.xml; Size: 8,331 bytes; and Date of Creation: Aug. 20, 2025) is herein incorporated by reference in its entirety.

### BACKGROUND OF THE INVENTION

Biotherapeutic proteins are vital medicines for a wide range of diseases with extracellular targets (Dumontet et al., Nature Reviews Drug Discovery 2023, 22 (8):641-661; Li et al., Nature Reviews Drug Discovery 2023, 22 (6): 449-475; Oostindie et al., Nature Reviews Drug Discovery 2022, 21 (10): 715-735; and Swain et al., Nature Reviews Drug Discovery 2023, 22 (2): 101-126). These protein-based drugs, however, sometimes acquire deleterious modifications during production and storage that can impact safety and efficacy (Wei et al., Drug Discovery Today 2014, 19 (1): 95-102; Yang et al., Mabs 2023, 15 (1): p. 2197668; and Federici et al. Biologicals 2013, 41 (3): 131-147).

For each drug, the production, formulation, and storage conditions must be optimized for stability to ensure molecular fidelity. Numerous assays are implemented to monitor their quality; however, only peptide mapping generates site specific modification levels used to identify critical quality attributes (CQAs). Peptide mapping is especially powerful for the analysis of monoclonal antibodies (mAbs) because their sequences typically only differ from one another in specific regions (Federici et al., Biologicals 2013, 41 (3): 131-147; and Lund et al., Journal of Pharmaceutical Sciences 2021, 110 (2): 619-626). Understanding the specific residues that are prone to modifications that affect efficacy is essential for all stages of mAbs development from discovery, through development and formulation, to production.

Conventional peptide mapping entails liquid chromatography-mass spectrometry (LC-MS) analysis of peptides enzymatically produced from the target protein (see FIG. 1). Typically, peptides composing the complete sequence and common chemical and biological modifications are detected and analyzed. The mass spectrometry (MS) signals for the modified and unmodified (i.e., native) peptides are compared to generate and estimate the modification stoichiometry. These measurements can be correlated with bioactivity/binding analyses to inform strategies for production, formulation, and storage of biotherapeutic proteins to specifically protect the critical amino acids. Accordingly, demand for peptide mapping analysis is growing substantially.

However, collection of peptide mapping data is cumbersome, complicated, and expensive, and often requires long data collection times and tedious data analyses to identify the amino acid sequences of therapeutic proteins and to detect changes to the amino acids. For example, aspartate and asparagine are able to be converted into isoaspartate (see FIG. 3) through reactions that may occur during the production or storage of therapeutic proteins, and which may compromise the safety or efficacy of such therapeutic proteins. Additionally, aspartate and isoaspartate have the same mass and therefore cannot be distinguished from each other using just MS (aspartate and isoaspartate are the ionic forms of aspartic acid and isoaspartic acid, respectively). Accordingly, detecting the conversion of aspartate and asparagine residues to isoaspartate in mAbs and other therapeutic proteins typically requires additional separation prior to MS analysis. Conventional peptide mapping methods therefore use an extended LC gradient (up to 120 minutes) in order to separate peptides containing isoaspartate from peptides containing aspartate. Consequently, many peptide mapping experiments often ignore critical modifications involving isoaspartate.

Accordingly, what is needed are improved methods and kits for the analysis and characterization of proteins and other polypeptides having isoaspartate, aspartate, and asparagine residues.

### SUMMARY OF THE INVENTION

The present invention provides methods and compositions for detecting isoaspartate residues in polypeptides using mass spectrometry (MS), and for distinguishing isoaspartate residues from aspartate and/or asparagine residues in a polypeptide using MS analysis.

In an aspect of the invention, polypeptides containing one or more isoaspartate residues, or suspected of containing one or more isoaspartate residues, are treated with L-isoaspartyl methyltransferase (PIMT), which is able to selectively modify isoaspartate (i.e., modify isoaspartate without affecting aspartate or asparagine residues) and impart a mass change to the isoaspartate residue. The polypeptide is then analyzed using MS, where a mass change caused by the PIMT reaction is detected and characterized. Preferably, methods of the present invention are able to be performed without the use of liquid chromatography (LC) or other similar separation techniques prior to MS analysis.

In an embodiment, the present invention provides a method of analyzing a first polypeptide having one or more amino acid residues of isoaspartate, aspartate, asparagine, or any combination thereof, where each isoaspartate residue, if present, has a first mass. The first polypeptide is contacted with a composition comprising protein L-isoaspartyl methyltransferase (PIMT), where the PIMT selectively modifies one or more isoaspartate residues, thereby generating a modified polypeptide comprising one or more modified isoaspartate residues. Each of the one or more modified isoaspartate residues has a different mass than the first mass of the one or more amino acid residues of isoaspartate. The method also comprises the steps of: analyzing the modified polypeptide using a mass spectrometer device, obtaining mass spectrometry data on the modified polypeptide, where the mass spectrometry data comprises a mass-to-charge ratio of the modified polypeptide and/or one or more fragment ions thereof; and characterizing the presence and/or amount of modified isoaspartate residues in the modified polypeptide based on the mass spectrometry data. Optionally, the mass spectrometry data comprises the signal intensities of the polypeptides and/or fragments thereof measured by the mass spectrometer device.

At physiological pH (pH ˜7), aspartic acid is deprotonated and becomes aspartate (similarly, isoaspartic acid becomes isoaspartate). However, when electrospray ionization is performed, polypeptide samples are often acidified to approximately pH 2, which is lower than the pH required for the formation of aspartic acid. In an embodiment, the PIMT reaction is performed at a pH such that the residues exist as the deprotonated forms (aspartate and isoaspartate). In an embodiment, where the subsequent mass spectrometry analysis, including electrospray ionization, is performed at around pH 3 or less, aspartate and isoaspartate residues predominantly take the protonated forms of aspartic acid and isoaspartic acid, which are one Dalton heavier than aspartate and isoaspartate. In an alternative embodiment, where the mass spectrometry analysis, including electrospray ionization, is performed at around pH 4 or greater, the residues may exist as predominantly aspartate and isoaspartate residues. In an embodiment, polypeptides having amino acid residues of isoaspartate and/or aspartate are intended to encompass polypeptides having the corresponding protonated isoapsartic acid and aspartic acid residues under acidic conditions.

PIMT is able to convert isoaspartate to form the corresponding O-methyl ester (e.g., O-methyl isoaspartate), and the O-methyl isoaspartate itself may be converted to a succinimide intermediate as illustrated in FIG. 4 (see Yang et al., Electrophoresis 2010, 31 (11): 1764-1772). In an embodiment, the O-methyl ester has a mass that is approximately 14-15 Da greater than the isoapartic acid/isoaspartate residue (14 Da greater than isoaspartic acid and 15 Da greater than the deprotonated isoaspartate). The succinimide intermediate has a mass that is approximately 17-18 Da less than the isoapartic acid/isoaspartate residue (18 Da less than isoaspartic acid and 17 Da less than the deprotonated isoaspartate).

Accordingly, in an embodiment, contacting a polypeptide having one or more isoaspartate residues with PIMT will generate a modified polypeptide where the one or more modified isoaspartate residues comprise one or more O-methyl isoaspartate residues and/or one or more succinimide residues thereof. Preferably, the one or more modified isoaspartate residues comprise one or more O-methyl isoaspartate residues. The modified polypeptide may then be analyzed by MS to detect the mass change from the isoaspartate residue to the modified residue. Optionally, the polypeptide will have multiple isoaspartate residues and treating the polypeptide with PIMT will modify and impart a mass change to each isoaspartate residue. Preferably, each of the one or more modified isoaspartate residues has a mass that differs from the first mass by a pre-selected amount. For example, each of the one or more modified isoaspartate residues has a second mass that is approximately 14-15 Da greater or 17-18 Da less than the first mass.

In an embodiment, the characterizing step comprises confirming a presence of modified isoaspartate residues in the modified polypeptide. Optionally, the characterizing step further comprises determining a ratio of modified isoaspartate residues in the modified polypeptide to unmodified aspartate residues in the modified polypeptide, or a ratio of modified isoaspartate residues in the modified polypeptide to unmodified aspartate residues in an unmodified polypeptide (i.e., a polypeptide whose mass does not undergo a change after being contacted with the composition comprising PIMT).

Optionally, the characterizing step comprises determining a quantitative amount of modified isoaspartate residues and/or unmodified aspartate residues in the modified polypeptide or in an unmodified polypeptide. For example, in an embodiment, the mass spectrometry data comprises signal intensity at one or more mass-to-charge ranges corresponding to modified isoaspartate residues and/or unmodified aspartate residues in the modified polypeptide, and determining the quantitative amount of modified isoaspartate residues and/or unmodified aspartate residues comprises comparing the signal intensity from the modified polypeptide to a signal intensity of a peptide standard comprising a known amount of isoaspartate residues and/or aspartate residues.

Optionally, the characterizing step comprises determining one or more amino acid positions in the amino acid sequence of the modified polypeptide that contain modified isoaspartate residues. For example, the amino acid positions containing the modified isoaspartate residues may be determined using tandem mass spectrometry (MS/MS or MS2). In an embodiment, determining one or more amino acid positions that contain the modified isoaspartate residues comprises the steps of: generating a first distribution of precursor ions from the modified polypeptide, fragmenting a portion of the precursor ions, thereby generating fragment ions, and measuring mass-to-charge ratios of the fragment ions. Optionally, determining the one or more amino acid positions that contain the modified isoaspartate residues further comprises the steps of measuring the signal intensities of the fragment ions.

The mass-to-charge ratios of the fragment ions are then, optionally, compared to fragment ions from peptide standards or known polypeptides that do not contain isoaspartate residues. In an embodiment, determining the amino acid positions containing the modified isoaspartate residues comprises the steps of: generating a first distribution of precursor ions from an unmodified polypeptide whose mass does not undergo a change after being contacted with the composition comprising PIMT, fragmenting a portion of the precursor ions from the unmodified polypeptide, thereby generating fragment ions from the unmodified polypeptide, and measuring mass-to-charge ratios of the fragment ions from the unmodified polypeptide. Optionally, the signal intensities of the fragment ions are also measured. The mass-to-charge ratios (and optionally the signal intensities) of fragment ions from the modified polypeptide may then be compared to the mass-to-charge fragment ions from the modified polypeptide to determine which amino acid position contain a mass difference.

Preferably, the polypeptide analyzed by the present invention is a therapeutic polypeptide that is part of a therapeutic drug or protein, or is an antibody, including but not limited to a monoclonal antibody (mAbs). As used herein, an antibody may refer to a complete antibody, or one or more polypeptide chains (i.e., one or more heavy chains or light chains) making up the antibody. In an embodiment, the analyzed polypeptide is a portion of a therapeutic polypeptide or antibody having or suspected of having one or more aspartate residues and/or asparagine residues modified to be isoaspartate residues. Optionally, the mass spectrometry data from the analyzed polypeptide is compared to a peptide standard or to a therapeutic polypeptide or antibody that is known not to contain any isoaspartate residues.

O-methyl isoaspartate may be converted to a succinimide residue at neutral or high pH values. However, the formation of the succinimide residues is able to be reduced or prevented by controlling the pH of the polypeptide after it has been contacted and modified by the PIMT composition. In an embodiment, the modified polypeptide is maintained in an environment having a pH of 8 or less, preferably a pH of 7 or less, a pH of 6 or less, a pH of 5 or less, a pH of 4 or less, or a pH of 3 or less. In an embodiment, formic acid is added to the modified polypeptide prior to MS analysis.

By selectively modifying the mass of isoaspartate without modifying aspartate, the presence of modified isoaspartate residues in a polypeptide may be determined through MS without performing a liquid chromatography step. For example, in an embodiment, the method comprises denaturing the modified polypeptide, treating the denatured polypeptide with a reducing agent, treating the reduced polypeptide with a first solution causing the reduced polypeptide to precipitate out of the first solution, redissolved the polypeptide precipitate in a second solution, and injecting the redissolved polypeptide into the ion source of the mass spectrometer device. As a result, the redissolved polypeptide may be injected into the mass spectrometry device without performing a liquid chromatography step, an additional salt removal step, and/or additional separation step.

Additionally, the polypeptide may be digested before or after being contacted with the PIMT composition. In an embodiment, the polypeptide is digested prior to contacting the polypeptide with the PIMT composition thereby generating two or more digestion fragments. Alternatively, the two or more digestion fragments are generated by digesting the modified polypeptide after contacting the first polypeptide with the PIMT composition and prior to obtaining mass spectrometry data. Digestion of the polypeptides may be performed using one or more digestion enzymes or chemical agents as is known in the art. Optionally, in embodiments where the polypeptide is digested prior to contact with the PIMT composition, the two or more digestion fragments, or the mixture or solution containing the two or more digestion fragments, are contacted with one or more inactivation agents able to inactivate the one or more digestion enzymes or chemical agents. For example, the one or more inactivation agents are able to inactivate a digestion enzyme, such as trypsin, so that the digestion enzyme does not affect the PIMT enzyme. In an alternative embodiment, the two or more digestion fragments, or the mixture or solution containing the two or more digestion fragments, are washed to remove the one or more digestion enzymes or chemical agents prior to the addition of PIMT.

In an embodiment, the PIMT is a modified PIMT. Preferably, the modified PIMT has an amino acid sequence having at least 85%, 90%, 95%, or 99% sequence identity to an unmodified human PIMT. Preferably the modified PIMT has an amino acid sequence having at least 85%, 90%, 95%, or 99% sequence identity to SEQ ID NO: 7. In an embodiment, the PIMT is a modified PIMT having increased resistance to enzymatic digestion, including but not limited to trypsin digestion, such as by modifying the N-terminus and/or C-terminus (such as through acetylation, amidation, or other modifications), modifying the peptide backbone (such as through methylating or modifying bonds in the peptide backbone), or combinations thereof. In an embodiment, the PIMT is a modified PIMT having increased resistance to enzymatic digestion, including but not limited to trypsin digestion, such as by inserting or substituting amino acids with non-canonical side chains into the PIMT, inserting one or more amino acids (such as proline) into the PIMT, or combinations thereof.

Optionally, liquid chromatography is performed on the modified polypeptide prior to obtaining mass spectrometry data on the modified polypeptide. In an embodiment, liquid chromatography is performed on the modified polypeptide to obtain chromatography data, including but not limited to, times of when the modified polypeptide elutes from a chromatography column. The chromatography data is then analyzed to determine if the modified polypeptide has an elution time that is within a time range corresponding to an unmodified polypeptide having an identical amino acid sequence except with aspartate residues and/or asparagine residues instead of isoaspartate residues.

In an embodiment, S-adenosylmethionine (SAM) is a co-substrate for the PIMT and isoaspartate reactions that form the O-methyl ester, where PIMT is also able to convert SAM to S-adenosyl-homocysteine (SAH) concurrent with the conversion of isoaspartate to O-methyl isoaspartate. Both SAM (399.15 m/z) and SAH (385.13 m/z) are able to be directly detected by MS. As a result, the conversion reaction of isoaspartate to the O-methyl isoaspartate is able to be monitored by measuring the presence of SAH in the sample. For example, in an embodiment, the method further comprises contacting a first sample comprising the first polypeptide with a pre-selected amount of SAM concurrently with or prior to contacting the first sample with the PIMT composition, where the PIMT composition converts SAM to SAH. Mass spectrometry data is then optionally obtained from the first sample by measuring mass spectrometry signal intensity at a mass-to-charge range corresponding to SAH, thereby obtaining a first SAH signal intensity, and comparing the first SAH signal intensity to a second SAH signal intensity from a calibration sample comprising a known amount of SAH. The amount of SAH generated by the PIMT composition in the sample may then be determined and used to calculate or confirm the amount of O-methyl isoaspartate generated in the sample. Optionally, the calibration sample comprises isotopically labeled SAH.

In an embodiment, one or more peptide standards are added and analyzed with the first polypeptide. Optionally, each of the one or more peptide standards has the same amino acid sequence as a polypeptide of interest and has a known amount of isoaspartate residues. In a further embodiment, mass spectrometry data is obtained from the modified polypeptide and compared with mass spectrometry data obtained from the one or more peptide standards treated with the PIMT composition. Preferably, the mass spectrometry data comprises signal intensity at one or more mass-to-charge ranges corresponding to modified isoaspartate residues in the modified polypeptide and signal intensity at one or more mass-to-charge ranges corresponding to modified isoaspartate residues in the one or more peptide standards. The amount of modified isoaspartate residues in the modified polypeptide may then be calculated by comparing the signal intensities. In an embodiment, the one or more peptide standards are isotopically labeled peptide standards, which optionally allows the position of the modified residues to be determined.

In an embodiment, the present invention provides a composition, and kits thereof, for analyzing a first polypeptide having one or more amino acid residues of isoaspartate, aspartate, asparagine, or any combination thereof, where the composition comprising: a) protein L-isoaspartyl methyltransferase (PIMT); b) one or more peptide standards, where each of the one or more peptide standards has a known quantity of isoaspartate residues; and c) a buffer solution. The buffer solution can be any buffer solution suitable for use with performing mass spectrometer as is known in the art, including but not limited to ammonium acetate. Optionally, the composition comprises one or more acids or acidic solutions, including but not limited to formic acid, able to maintain a solution containing the modified peptide at a pH less than 8 (preferably less at a pH than 7). Optionally, the composition also comprises one or more digestion enzymes or chemical agents used to digest peptides. Preferably, the composition does not contain any detergents and/or additional salts, and allows for direct analysis of the polypeptide and allows a user to directly infuse samples into the MS device. In an embodiment, the composition further comprises S-adenosylmethionine (SAM) and a calibration sample containing a standard peptide.

In an embodiment, each of the one or more peptide standards comprises either 0% or 100% isoaspartate residues relative to a total amount of isoaspartate and aspartate residues. Optionally, each peptide standard that contains at least one isoaspartate residue contains only one isoaspartate residue per peptide. In a further embodiment, the composition comprises one peptide standard having no isoaspartate residues and one peptide standard having the same amino acid sequence but where all aspartate residues are replaced with isoaspartate residues. The amount of isoaspartate residues in the modified polypeptide is able to be calculated by comparing MS data from the modified peptide with the peptide standards containing either no isoaspartate residues or 100% isoaspartate residues (relative to a total amount of isoaspartate and aspartate residues).

In an embodiment, the one or more peptide standards are isotopically labeled. Optionally, the isotopic label may be present at or near the amino acid position where the isoaspartate residue is located in the modified polypeptide. Preferably, the isotopically labeled peptide standards have at least 90% sequence identity, at least 95% sequence identity, at least 98% sequence identity, or at least 99% sequence identity to the first polypeptide except where no aspartate or asparagine residues have been substituted or replaced. In an embodiment, the isotopically labeled peptide standards have the identical sequence as the first polypeptide except where no aspartate or asparagine residues have been substituted or replaced. Optionally, the isotopically labeled standards comprise a variety of peptide sequences that are expected or could be modified from the first polypeptide to contain one or more isoaspartic acid modifications.

### DESCRIPTION OF THE DRAWINGS

FIG. 1 shows an overview of current peptide mapping and technology.

FIG. 2 illustrates technology (see PCT/US24/17865 to Coon et al.) to accelerate peptide mapping by 100-fold using direct infusion that does not require liquid chromatography.

FIG. 3 illustrates reactions forming isoaspartate from aspartate and asparagine through a succinimide intermediate, as well as the chemical structure of S-adenosylmethionine (SAM) and S-adenosyl-homocysteine (SAH).

FIG. 4 illustrates reactions forming isoaspartate from aspartate as well as PIMT reaction forming O-methyl isoaspartate from isoaspartate.

FIG. 5 provides a table of two peptide standards containing isoaspartate residues, DSIP (WAGGDiASGE—SEQ ID NO:4) and VAAK (YVYVADiVAAK—SEQ ID NO:5) mixed with NIST mAb peptides in various ratios. The samples, except for a control, were treated with PIMT.

FIG. 6 shows correlation plots (non-log) of measured versus known isoaspartate amounts in samples containing the DSIP (SEQ ID NO: 4) and VAAK (SEQ ID NO: 5) protein standards from FIG. 5 with and without a NISTmAb peptide background.

FIG. 7 shows correlation plots (log 2) of measured versus known isoaspartate amounts in samples containing the DSIP (SEQ ID NO: 4) and VAAK (SEQ ID NO: 5) protein standards from FIG. 5 with and without a NISTmAb peptide background.

FIG. 8 shows correlation plots of measured isoaspartate amounts in samples containing a DSIP (SEQ ID NO: 4) protein standard with and without a NISTmAb background, and samples containing a VAAK (SEQ ID NO: 5) peptide standard with and without a NISTmAb background.

FIG. 9 shows correlation plots of measured isoaspartate amounts between the DSIP (SEQ ID NO: 4) and VAAK (SEQ ID NO: 5) peptide standards.

FIG. 10 is a table providing the MS signal intensity of S-adenosyl-homocysteine (SAH) and the O-methyl ester resulting from a polypeptide starting with various known amounts of isoaspartate.

FIG. 11 shows correlation plots (non-log and log 2) of measured SAH and the O-methyl ester amounts from FIG. 10.

FIGS. 12 and 13 show PIMT labeling performed using either DSIP that does not contain the isoaspartate residue (SEQ ID NO:1) (FIG. 12) or IsoAsp-DSIP (SEQ ID NO:4) (FIG. 13) as a substrate. Sequences, if applicable, and accurate monoisotopic masses of singly charged ions are as follows: SAM, 399.149; SAH, 385.129; DSIP (WAGGDASGE—SEQ ID NO:1), 849.3373; IsoAsp-DSIP (WAGGisoDASGE—SEQ ID NO:4), 849.3373; Succinimide-variants, 831.3268; Methylester-variants, 863.3530.

FIG. 14 shows exemplary MS1 spectra for a PIMT reaction with the IsoAsp peptide DSIP (WAGGDiASGE—SEQ ID NO:4). Zoom-in sections focus on SAM and SAH (380-405 m/z) or on DSIP, including the succinimide and unmodified form (succinimide, 831.327; unmodified, 849.337; methylester, 863.353).

FIG. 15 shows MS1 spectra for additional reactions with reaction composition identical to FIG. 14 but with different reaction times and temperatures.

FIG. 16 shows the effect pH has stability of reaction products for PIMT reactions using the DSIP (WAGGDiASGE—SEQ ID NO:4), VAAK (YVYVADiVAAK—SEQ ID NO:5), and PennyK (GFYPSDIAVEWESDiGQPENNYK—SEQ ID NO:6) IsoAsp peptides. The peptides were included in the same reaction at a concentration of 1.33 μM each (n=3). In the bar graphs, the unmodified peptide form is the left bar, the succinimide form is the middle bar, and the methylester form is the right bar.

FIG. 17 shows extracted-ion chromatograms (XICs) from an LC-MS experiment, presenting leftover unmodified peaks from IsoAsp PennyK (SEQ ID NO:6) and IsoAsp VAAK (SEQ ID NO: 5) PIMT reactions from FIG. 16. As seen in the XICs, the majority of the unmodified peaks are in the IsoAsp form.

FIG. 18 shows the retention time for the Asp (not modified) PENNYK isoform (SEQ ID NO: 3), IsoAsp-PENNYK (SEQ ID NO: 6) isoform, Asp (not modified) VAAK isoform (SEQ ID NO: 2), and IsoAsp-VAAK isoform (SEQ ID NO: 5) from FIG. 17.

FIG. 19 shows resulting SAH intensity for PIMT reactions performed on the IsoAsp-DSIP (SEQ ID NO: 4), IsoAsp-VAAK (SEQ ID NO:5), and IsoAsp-PennyK (SEQ ID NO:6) peptides individually. Resulting SAH levels remained very similar, demonstrating that SAH percentages are a good indicator for overall IsoAsp levels in the sample.

FIG. 20 shows resulting SAH intensity for a pooled sample containing DSIP (SEQ ID NO: 1), VAAK (SEQ ID NO: 2) and PennyK (SEQ ID NO: 3) subjected to the PIMT reaction using different input amounts of SAM in order to determine the impact of SAM concentration on PIMT efficiency.

FIG. 21 shows reaction products of a PIMT reaction that was adjusted with 0.5% formic acid, followed by incubation for indicated amounts of time at 8° C. prior to DI-MS analysis.

FIG. 22 shows reaction products of a PIMT reaction where methanol (MeOH) was added to the reaction in order to stabilize some of the reaction products and generally push the reaction equilibrium more into the direction of the reaction products.

FIG. 23 shows a PIMT reaction similar to FIG. 22 where the concentrations of MeOH were varied to determine PIMT enzymatic efficiency.

FIG. 24 shows a PIMT reaction similar to FIG. 22 where ethanol (EtOH) and acetonitrile (ACN) were substituted for MeOH.

FIG. 25 shows reaction completeness of PIMT reactions using different peptide concentrations as a reaction input. Reaction efficiencies for IsoAsp-DSIP (SEQ ID NO:4) and IsoAsp-VAAK (SEQ ID NO:5), even at concentrations of 50 μM (=16.66 μM per peptide) were excellent. For IsoAsp-PennyK (SEQ ID NO:6), residual amounts of the initial peptide remained which increased with higher peptide concentrations.

FIG. 26 shows PIMT reactions with varying reaction duration (2 hrs, 5 hrs, and overnight) and temperature (room temperature, 30° C., and 37° C.).

FIG. 27 shows an experimental layout of an IsoAsp dilution experiment, where the amount of each IsoAsp peptide decreases across samples but the overall peptide amount stays constant. Samples were either PIMT or Mock-treated and analyzed both via DI-MS and LC-MS to compare accuracy of DI-MS with LC-MS. DSIP (SEQ ID NO:1), IsoAsp-DSIP (SEQ ID NO:4), VAAK (SEQ ID NO: 2) IsoAsp-VAAK (SEQ ID NO:5), PennyK (SEQ ID NO: 3), and IsoAsp-PennyK (SEQ ID NO:6).

FIG. 28 shows the results of the experiment of FIG. 27 across varying concentrations. The correlation of known concentrations within the DI-MS (PIMT-treated) results was very high.

FIG. 29 shows the results of the experiment of FIG. 27 obtained using DI-MS (PIMT-treated) and LC-MS (PIMT-treated). The correlation between DI-MS and LC-mS was also high.

FIG. 30 shows the results of the experiment of FIG. 27 between DI-MS (PIMT-treated) and LC-MS (Mock-treated). Again, the correlation was very high.

### DETAILED DESCRIPTION OF THE INVENTION

### Definitions

In general the terms and phrases used herein have their art-recognized meaning, which can be found by reference to standard texts, journal references and contexts known to those skilled in the art. The following definitions are provided to clarify their specific use in the context of the invention.

The term “protein” refers to a class of compounds comprising a sequence of amino acid residues connected via peptide bonds to form one or more polypeptide chains and/or modified polypeptide chains. Proteins can be modified by naturally occurring processes such as post-translational modifications or co-translational modifications.

The terms “peptide” and “polypeptide” are used synonymously in the present description, and refer to a class of compounds composed of amino acid residues chemically bonded together by amide bonds (or peptide bonds). Peptides and polypeptides are polymeric compounds comprising at least two amino acid residues or modified amino acid residues. Modifications can be naturally occurring or non-naturally occurring, such as modifications generated by chemical synthesis. Peptides and polypeptides can be generated by substantially complete digestion or by partial digestion of proteins.

The terms “residue” and “amino acid residue” refer to an amino acid that is part of a polypeptide or protein and forms a peptide bond with at least one other amino acid. Isoaspartate, aspartate, asparagine, and O-methyl isoaspartate residues are illustrated below (aspartate and isoaspartate are the ionic forms of aspartic acid and isoaspartic acid), with each residue forming two peptide bonds although it should be noted that each residue could form a single peptide bond at either the amino group or carboxyl group:

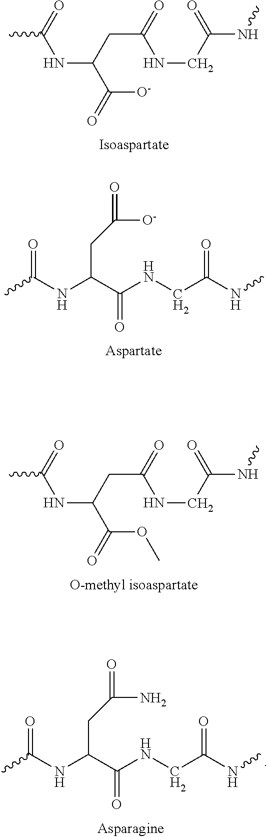

As used herein, the term “mass spectrometry” (MS) refers to an analytical technique for the determination of the elemental composition of an analyte, including but not limited to polypeptides. The mass spectrometry principle consists of ionizing analytes to generate charged species (i.e., precursor ions) or species fragments (i.e., product ions) and measurement of their mass-to-charge ratios. Conducting a mass spectrometric analysis of an analyte results in the generation of mass spectrometry data relating to the mass-to-charge ratios of the analyte and analyte product ions. Mass spectrometry data corresponding to analyte ion and analyte ion fragments is presented in mass-to-charge (m/z) units representing the mass-to-charge ratios of the analyte precursor ions and/or analyte product ions. In tandem mass spectrometry (MS/MS or MS2), multiple rounds of mass spectrometry analysis are performed. For example, samples containing a mixture of polypeptides can be ionized and the resulting precursor ions separated according to their mass-to-charge ratio. Selected precursor ions can then be fragmented and further analyzed according to the mass-to-charge ratio of the product ions.

As used herein, the term “mass spectrometer” refers to a device which creates ions from a sample, separates the ions according to mass to charge ratios, and measures the abundance of each detected m/z peak. Because charge (z) can be calculated from the spectra, mass can be determined. Mass spectrometers include multistage mass spectrometers which fragment the mass-separated precursor ions and separate the product ions by mass-to-charge ratio one or more times. Multistage mass spectrometers include tandem mass spectrometers which fragment the mass-separated precursor ions and separate the product ions by mass-to-charge ratio once.

As used herein, the term “precursor ion” is used herein to refer to an ion which is produced during ionization stage of mass spectrometry analysis, including the MS1 ionization stage of MS/MS analysis. As used herein, the term “product ion” is used to refer to an ion which is produced during a fragmentation process of a precursor ion, including the MS2 ionization stage of MS/MS analysis.

As used herein, the term “mass-to-charge ratio” refers to the ratio of the mass of a species (i.e., a precursor ion or product ion) or to the charge state of a species. The term “m/z unit” refers to a measure of the mass to charge ratio. The Thomson unit (abbreviated as Th) is an example of an m/z unit and is defined as the absolute value of the ratio of the mass of an ion (in Daltons) to the charge of the ion (with respect to the elemental charge).

### Overview

Biotherapeutic proteins, including but not limited to antibodies, are vital medicines for a wide range of diseases with extracellular targets. These protein-based drugs, however, sometimes acquire deleterious modifications during production and storage that can negatively impact the safety and efficacy of the therapeutic proteins. For example, aspartate and asparagine residues may undergo isomerization and form isoaspartate residues. Numerous assays are often implemented to monitor the quality of such therapeutic peptides; however, currently only peptide mapping generates site specific modification levels used to identify critical quality attributes (CQAs). Peptide mapping is especially powerful for the analysis of monoclonal antibodies (mAbs) because their sequences typically only differ from one another in specific regions.

As of 2023, there were over 160 approved mAbs drugs with the therapeutic mAbs market estimated to exceed $200 billion. Furthermore, there is a surging market for global biosimilar mAbs production as many of the initial mAbs drugs are now coming off patent. This surge is expected to grow the mAbs biosimilar market to over $11 billion by 2027 (The Business Research Company, Biosimilars Monoclonal Antibody Market Report, 2024.). All stages of mAbs development rely heavily on peptide mapping. The first discovery stage evaluates mAbs production across numerous cell lines and antibody sequences. In the subsequent development stage, peptide mapping is used for process optimization, to access formulation stability, antibody tolerance to heat, pH, light, etc., and to examine lot production. Lastly, peptide mapping is heavily used in manufacturing and quality control. It is estimated that the global pharmaceutical industry will perform between 2 and 5 million peptide maps in 2024, and this number is certain to continue to rise with the continued investment and success of mAbs and other therapeutic drugs.

Collection of peptide mapping data, however, is both cumbersome and complicated, with some inquiries showing that collection of 16 peptide maps (or 16 samples) requires one day of LC-MS time and two days of expert analyst time to complete.

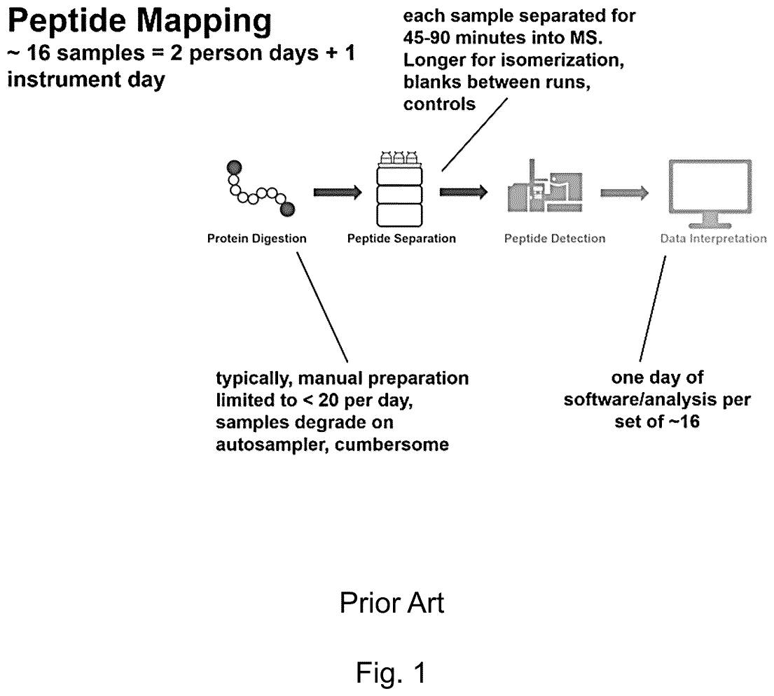

In conventional peptide mapping (illustrated in FIG. 1), proteins are digested into multiple constituent polypeptides by chemical or enzymatic reactions and are then separated, such as by liquid chromatography (LC). A reliable separation platform (i.e., an LC column) should be used that is able to deliver high resolution between hundreds of polypeptides resulting from the digestion. Conventional peptide separation requires about 45-90 minutes per sample, although longer times are required to separate polypeptides having isomerized amino acids residues (such as aspartate and isoaspartate). Following peptide separation, the polypeptides are analyzed using mass spectrometry (MS) to detect the polypeptides of interest. Using appropriate software, conventional peptide mapping systems are typically able to analyse between 15-20 samples per day (approximately 4,000 samples a year per machine) at a cost of over $400 per sample.

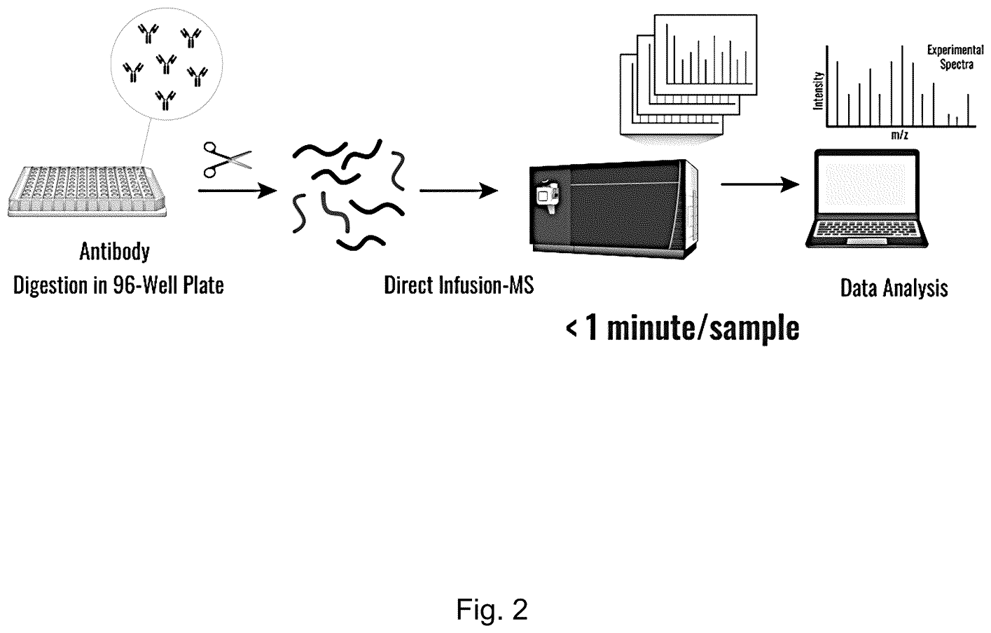

In order to improve speed and efficiency, additional methods (see PCT/US24/17865 to Coon et al.) have been developed for peptide mapping that directly analyzes digested antibodies and other therapeutic proteins through direct infusion into the mass spectrometry device without having to perform liquid chromatography. One such method (illustrated in FIG. 2) leverages a plate-based sample preparation compatible with direct infusion MS analysis and is capable of rapidly preparing and analyzing 96 samples within 8 hours (4 hours sample preparation and 4 hours of MS analysis). With the proper sample preparation method and high resolution MS, >95% of the mAbs sequence is able to be observed. This alone is useful in the rapid confirmation of the identity of the biotherapeutic protein. These methods also enable measurements of methionine and tryptophan oxidation, asparagine glycosylation, and, by addition of targeted MS/MS scans, site-specific deamidation. However, one limitation of peptide mapping methods that do not utilize LC separation is the inability to detect conversion of aspartate to isoaspartate.

### Detection of Isoaspartate

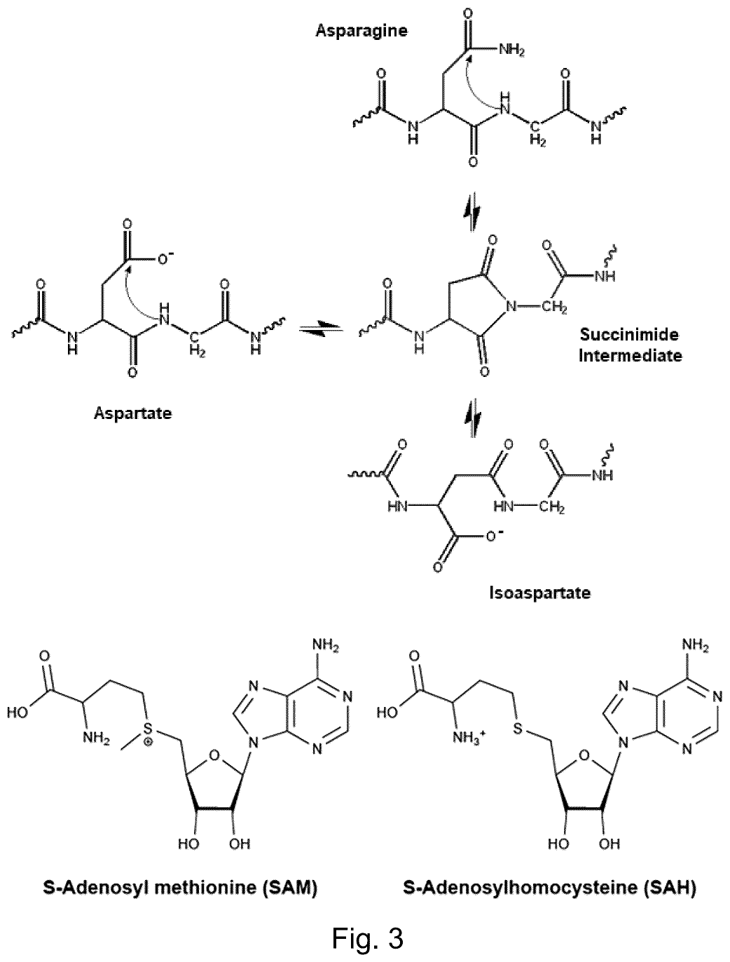

Aspartate and asparagine residues are able to form isoaspartate through a succinimide intermediate as shown in FIG. 3. Detection of the conversion of aspartate and asparagine residues to isoaspartate is an important measurement of quality and safety in mAbs and other therapeutic proteins. To detect conversions of aspartate residues to isoaspartate by conventional peptide mapping, the LC gradient is extended so that the peptides containing isoaspartate are separated from the unmodified peptides retaining the original amino acid residues. In current conventional methods, the LC gradient is extended from approximately 45 minutes up to 120 minutes, which further increases the time and cost to perform peptide mapping. Consequently, many peptide mapping experiments ignore modifications that result in the formation of isoaspartate.

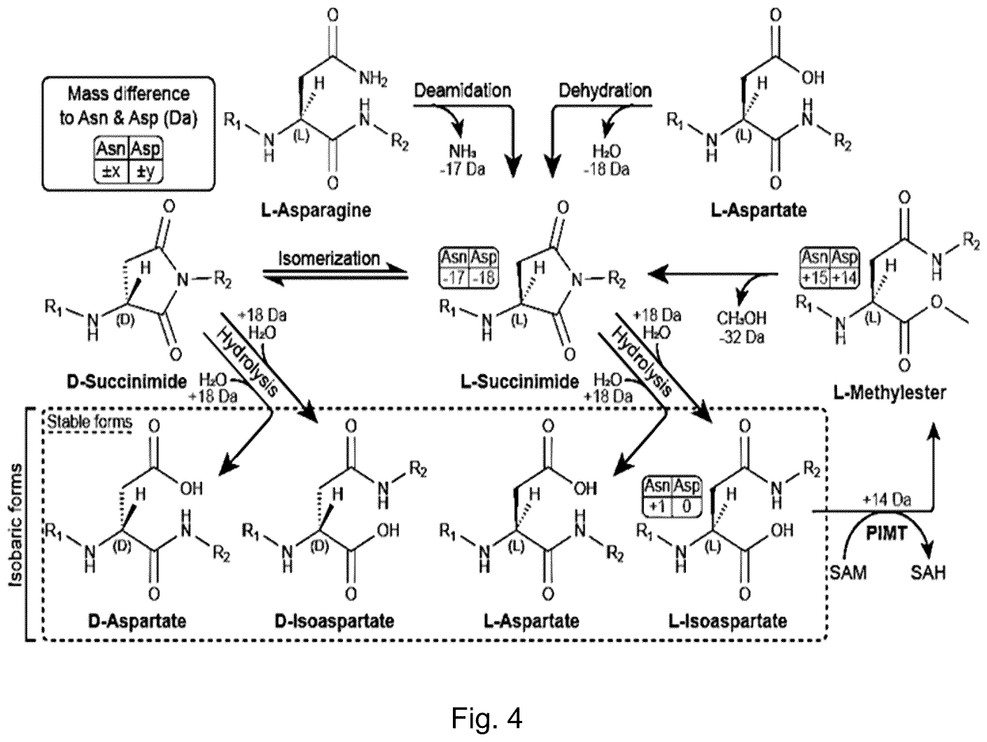

The present invention discloses a method for using the enzyme protein L-isoaspartyl methyltransferase (PIMT) to selectively convert isoaspartate into O-methyl isoaspartate while aspartate and asparagine residues are unaffected (see FIG. 4) (see also Hsiao et al., Journal of Pharmaceutical Sciences 2017, 106 (6): 1528-1537; Hooi et al., Mechanisms of Ageing and Development 2013, 134 (3-4): 103-109; Liu et al., Analytical Chemistry 2012, 84 (2): 1056-1062; Shimizu et al., Biological & Pharmaceutical Bulletin 2005, 28 (9): 1590-1596; and Yang et al., Electrophoresis 2010, 31 (11): 1764-1772).

Therapeutic proteins or other polypeptides having aspartate and/or asparagine residues suspected of having isomerized into isoaspartate residues are contacted with a composition comprising PIMT in order to generate a modified polypeptide having modified isoaspartate residues (i.e., O-methyl isoaspartate residues). Polypeptides having O-methyl isoaspartate residues are able to be easily distinguished from polypeptides having the corresponding aspartate and asparagine residues using MS without requiring LC separation. For example, the O-methyl isoaspartate has a mass 15 Da heavier than isoaspartate (14 Da heavier than aspartic acid), which is a mass difference that can be detected using the direct injection approach of PCT/US24/17865.

Additionally, as is also shown in FIG. 4, PIMT is able to convert isoaspartate residues into the O-methyl ester moiety with concomitant conversion of S-adenosylmethionine (SAM) to S-adenosyl-homocysteine (SAH) (Yang et al., Electrophoresis 2010, 31 (11): 1764-1772). Both SAM (SAM+ monoisotopic 399.15 m/z) and SAH (SAH+ monoisotopic 385.13 m/z) are able to be directly detected by MS (see FIG. 3). Therefore, measuring the presence of SAH in the sample is able to confirm the modification of isoaspartate to the O-methyl isoaspartate. Detection and characterization of the succinimide intermediate may also be incorporated into the overall calculation to improve precision.

In some environments, the O-methyl isoaspartate is not stable and a portion of the O-methyl isoaspartate can revert back to isoaspartate through the succinimide intermediate (see FIG. 4). This reaction, however, is pH dependent and the O-methyl isoaspartate product can be rendered stable by adjusting the pH value, such as maintaining a pH of 8 or less (Hsiao et al., Journal of Pharmaceutical Sciences 2017, 106 (6): 1528-1537).

### EXAMPLES

### Example 1—PIMT Labeling

Exemplary peptide standards Delta Sleep-Inducing Peptide (DSIP) and VAAK, a peptide derived from the human protein PON3, were prepared containing either one isoaspartate (isoAsp) residue or no isoaspartate residues. Reacting the DSIP and VAAK peptides that do not contain an IsoAsp residue with PIMT should not yield any peptide modifications or labeling. The amino acid sequence for each peptide standard was as follows:

DSIP Sequence:
(SEQ ID NO: 1)
WAGGDASGE,
IsoAspDSIP Sequence:
(SEQ ID NO: 4)
WAGG[isoAsp]ASGE,
VAAK Sequence:
(SEQ ID NO: 2)
YVYVADVAAK,
IsoAspVAAK Sequence:
(SEQ ID NO: 5)
YVYVA[isoAsp]VAAK,
PENNYK Sequence:
(SEQ ID NO: 3)
GFYPSDIAVEWESDGQPENNYK,
and
IsoAsP PENNYK Sequence:
(SEQ ID NO: 6)
GFYPSDIAVEWESDIGQPENNYK.

Stock solutions used in the reactions were prepared as follows: stock solution of each peptide standard: 100 pmol/μl in water; SAM stock solution: (1 mM SAM, 40 mM HCl); PIMT stock solution (provided by Promega Corporation): (0.53 mg/ml PIMT, 20 mM potassium phosphate pH 7.2, 1 mM EDTA, 1 mM DTT, 50% glycerol); and 10% formic acid. Concentrations of purified peptide mixtures are adjusted as required, depending on the type of peptide sample.

To perform the PIMT reaction for each sample, 86 μl of water was combined with 10 μl of 250 mM ammonium acetate, 1 μl of the SAM stock solution, 1 μl of a stock solution of a peptide standard, and 2 μl of the PIMT stock solution.

This reaction yields a sample volume of 100 μl having the following concentrations of the main components: 25 mM ammonium acetate, 10 pmol/μl of SAM, 1 pmol/μl peptide standard, and ˜1 μg of PIMT. The reaction solution was incubated for 2 h at either 30° C. or 37° C. Incubating the reaction at 30° C. leads to less succinimide production, whereas performing the reaction at 37° C. is more convenient for some applications since tryptic digestion is often conducted at 37° C. as well. After the 2 h incubation, a quench reaction is performed and a sample for mass spectrometry analysis was prepared by adding 10 μl of the 10% formic acid stock solution.

### Example 2—Analysis by LC-MS

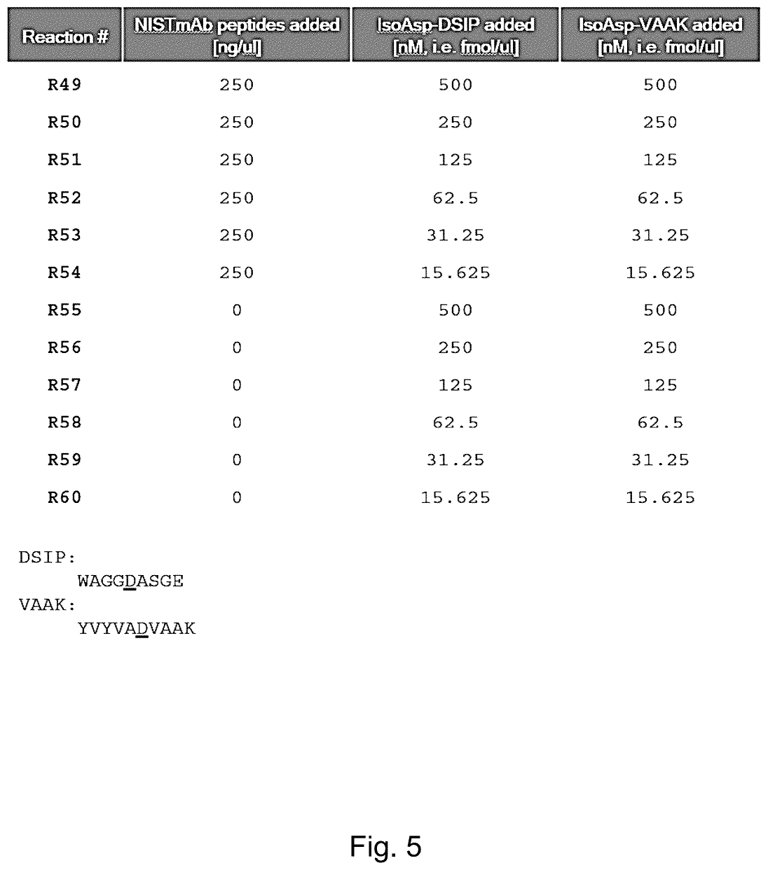

Although the methods of the present invention are able to be performed without the use of liquid chromatography, additional experiments may use liquid chromatography to provide additional data or to confirm the effectiveness of the PIMT labeling. In one such experiment, peptide standards, IsoAsp-DSIP (SEQ ID NO: 4) and IsoAsp-VAAK (SEQ ID NO: 5)) containing isoaspartate residues were mixed with NIST mAb peptides in various ratios, where the samples were treated with PIMT (see FIG. 5). Each sample was analyzed using LC-MS in technical duplicates on an Orbitrap Ascend using a 40 min total gradient and a DDA OT-IT method setup.

Each PIMT reaction was analyzed (=run) in duplicates. For each run, MS intensities of O-methyl versions for both IsoAsp-DSIP (SEQ ID NO: 4) and IsoAsp-VAAK (SEQ ID NO: 5) were obtained, and the O-methyl IsoAsp-DSIP intensities and O-methyl IsoAsp-VAAK intensities were averaged across duplicates. For both O-methyl IsoAsp-DSIP and O-methyl IsoAsp-VAAK, average values for the highest concentration (500 nM) were set to 100%→O-methyl IsoAsp measured [%]. For IsoAsp known, 500 nM were set to 100%.

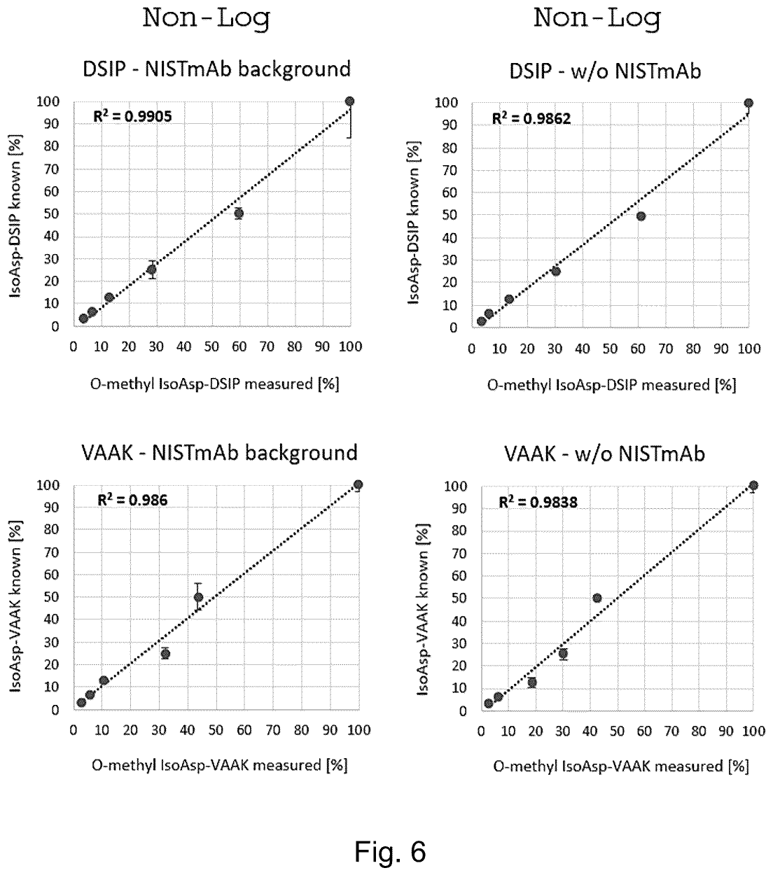

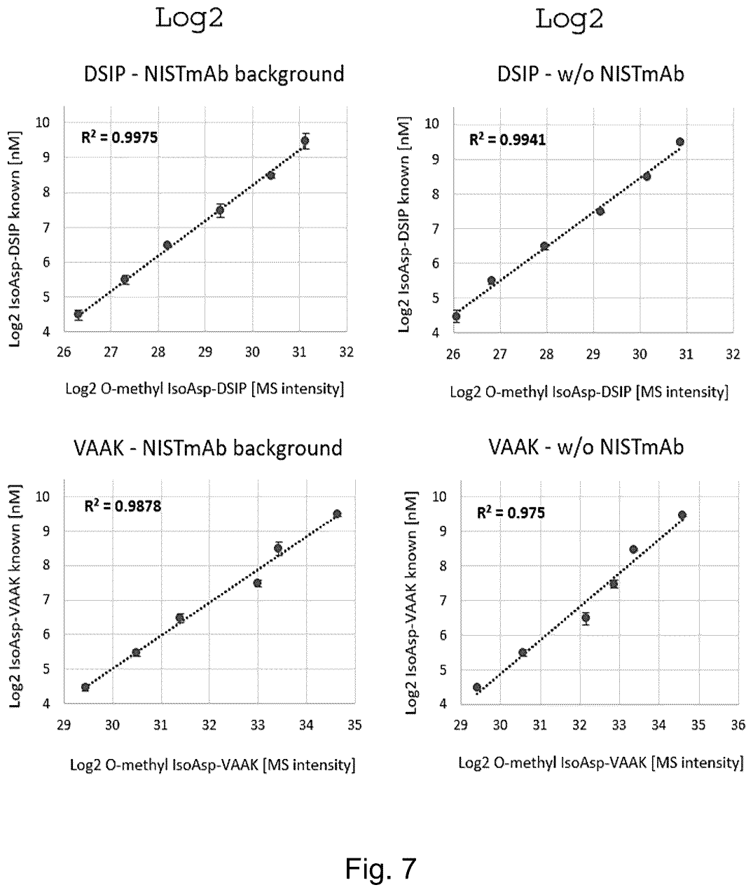

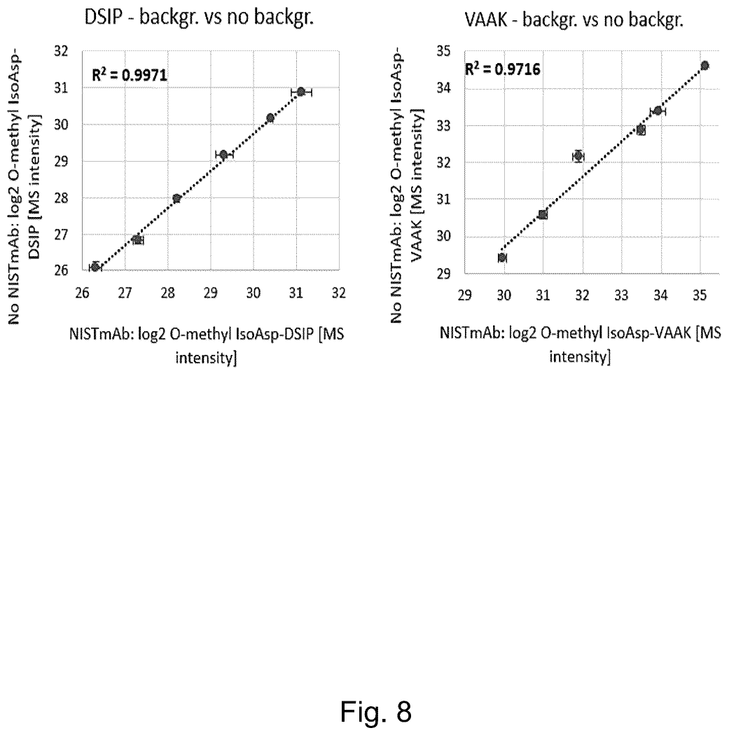

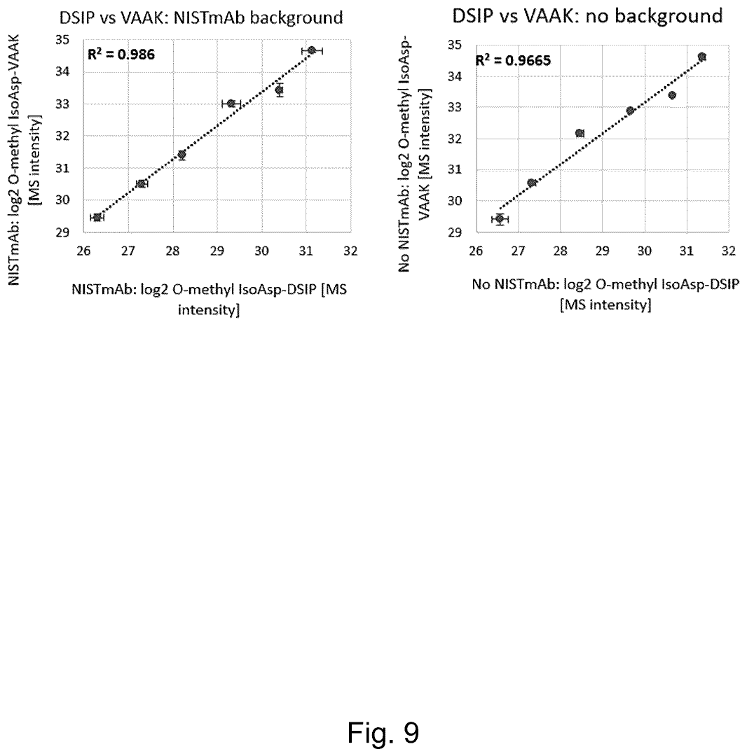

FIGS. 6 and 7 show correlation plots of measured vs known isoaspartate amounts in samples with and without the NISTmAb peptide background, and based on IsoAsp-DSIP (SEQ ID NO: 4) and IsoAsp-VAAK (SEQ ID NO: 5) conversion to the O-methylester variants. FIG. 8 shows correlation plots of measured isoaspartate amounts in samples containing a DSIP protein standard with and without a NISTmAb background, and samples containing a VAAK peptide standard with and without a NISTmAb background, and FIG. 9 shows correlation plots of measured isoaspartate amounts between the DSIP and VAAK peptide standards. Overall, these figures demonstrate that isoaspartate can be consistently detected across different polypeptides, different concentrations, and even in the presence of NISTmAb peptides.

For the Log 2 plots shown in FIG. 7, instead of using percentages, the underlying values were used, i.e. Log 2 transformed average MS intensities and the Log 2 nM concentrations. For plots shown in FIGS. 8 and 9, the same Log 2 transformed average MS intensities as shown in FIG. 7 were used.

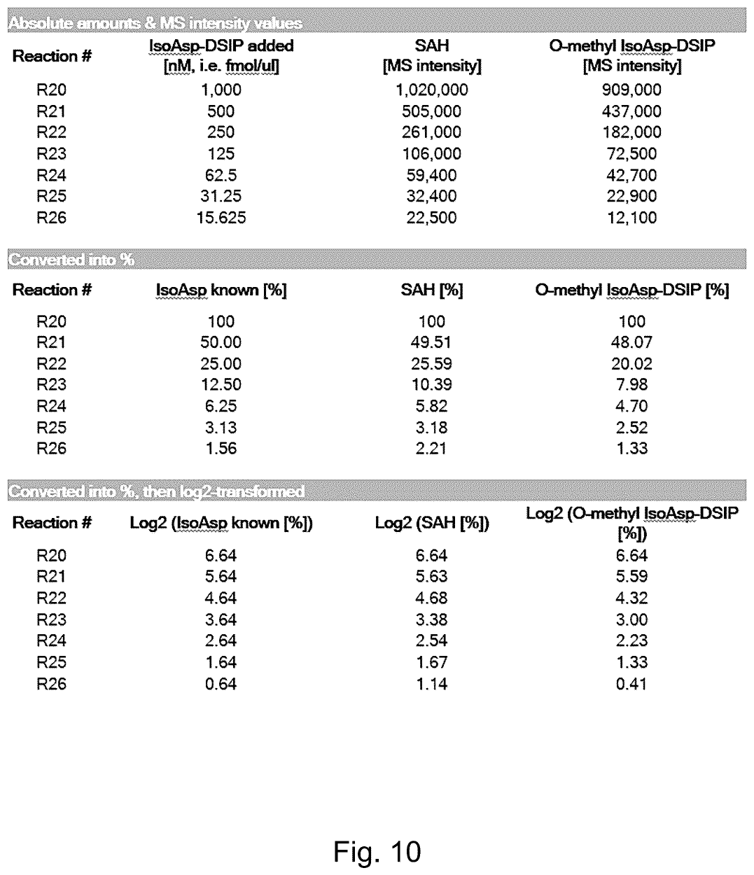

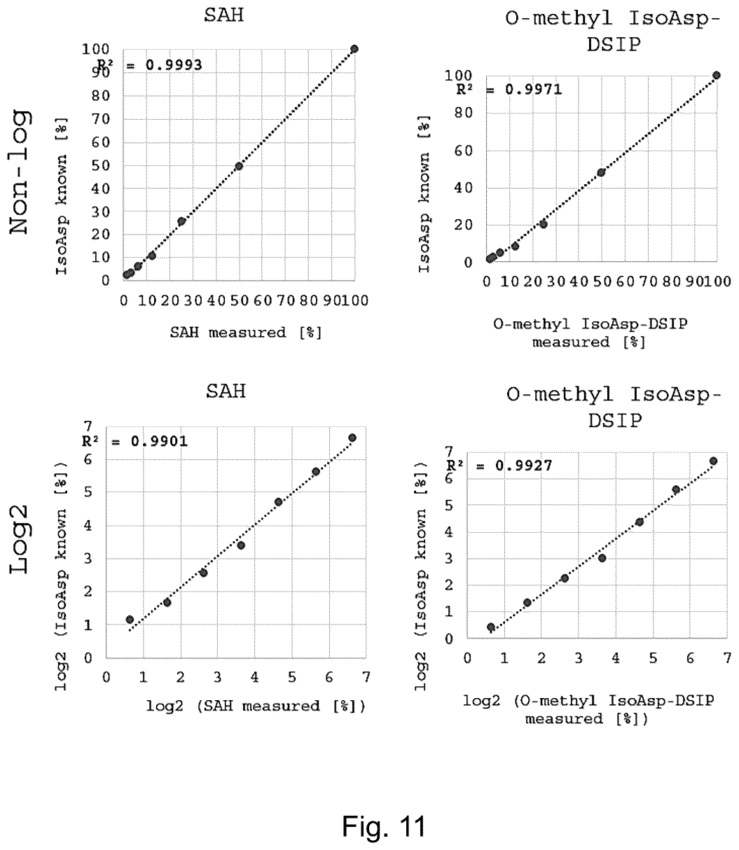

FIGS. 10 and 11 provide MS signal intensity and correlation plots of O-methyl IsoAsp-DSIP and S-adenosyl-homocysteine (SAH). IsoAsp-DSIP (SEQ ID NO: 4) was used as a substrate and the data was obtained by direct infusion, i.e., without LC-based peptide separation prior to mass spectrometry analysis. Overall, these figures demonstrate that SAH can also be consistently detected and measured across different concentrations in conjunction with the O-methyl ester.

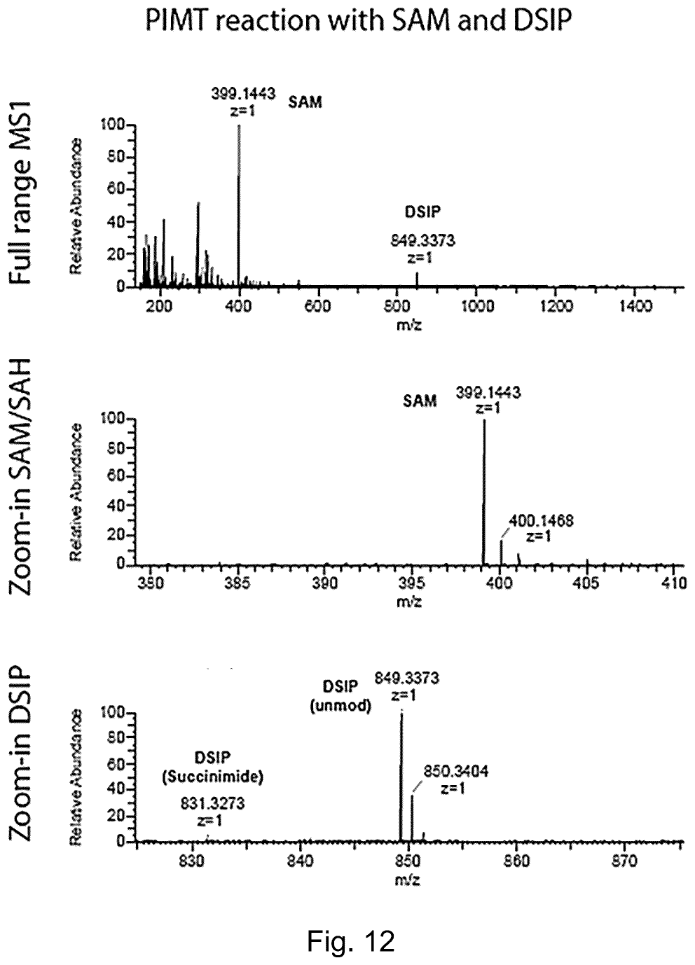

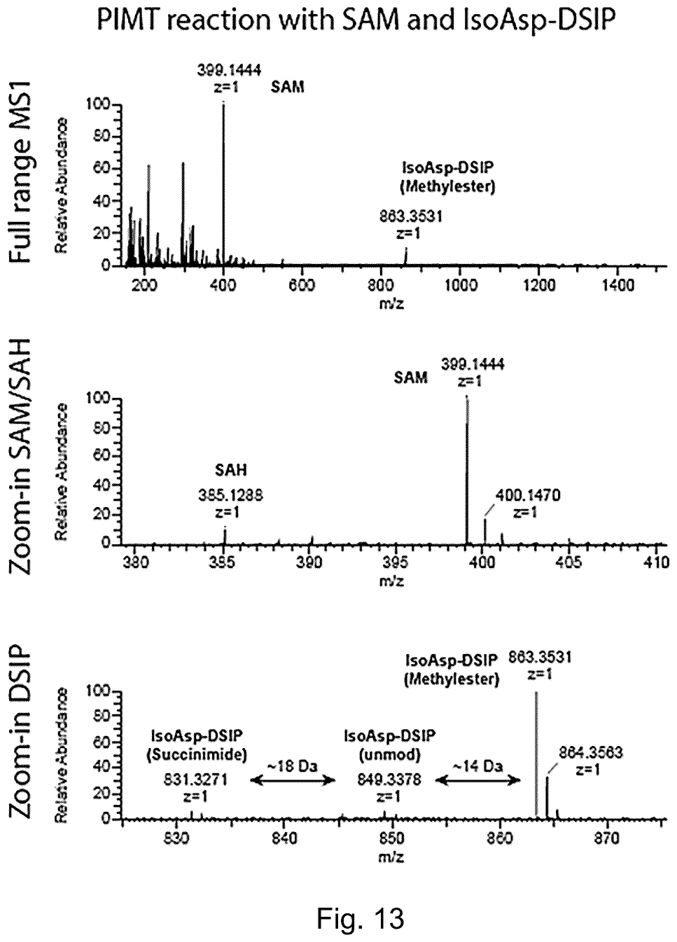

FIGS. 12 and 13 show PIMT labeling reaction performed using SAM and either DSIP (SEQ ID NO: 1) (FIG. 12) or IsoAsp-DSIP (SEQ ID NO: 4) (FIG. 13). Sequences, if applicable, and accurate monoisotopic masses of singly charged ions are as follows: SAM, 399.149; SAH, 385.129; DSIP (WAGGDASGE—SEQ ID NO: 1), 849.3373; IsoAsp-DSIP (WAGGisoDASGE—SEQ ID NO: 4), 849.3373; Succinimide-variants, 831.3268; Methylester-variants, 863.3530

Accordingly, the present invention reliably enables the identification of isoaspartate in therapeutic proteins using peptide mapping, including direct injection peptide mapping. While the conversion of aspartate and asparagine residues can be identified using LC-based methods, such methods often require extending the LC gradient time from 45 minutes (an already undesirable time period) to 120 minutes. Addition of this enzymatic PIMT-based conversion could be integrated into current direct-injection workflow with little to no effect on the analysis time, and can be supplemented with SAH measurements to improve reliability and precision. SAH measurements give a global view of the IsoAsp amount in the sample, which becomes relevant if sample contains more than one polypeptide that could potentially contain an IsoAsp residue.

### Example 3—Optimization of PIMT Labeling

In the reactions described below, peptides containing an isoaspartate (IsoAsp) residue were treated with PIMT using different experimental conditions in order to determine optimized conditions for the reaction. Such conditions included varying the pH of the reaction, the amount of SAM, the types of solvents used, concentration of the peptides, reaction time, and temperature.

In a typical workflow, the PIMT reaction was performed on the peptides, stopped with the addition of formic acid (FA), and the resulting products analyzed using LC-MS or direct infusion mass spectrometry (DI-MS). The amino acid sequence for each IsoAsp peptide was as follows (IsoAsp site indicated using the subscript “i”): IsoAsp-DSIP—WAGGDiASGE (SEQ ID NO: 4), IsoAsp-VAAK: YVYVADIVAAK (SEQ ID NO: 5), and IsoAsp-PennyK—GFYPSDIAVEWESDiGQPENNYK (SEQ ID NO: 6).

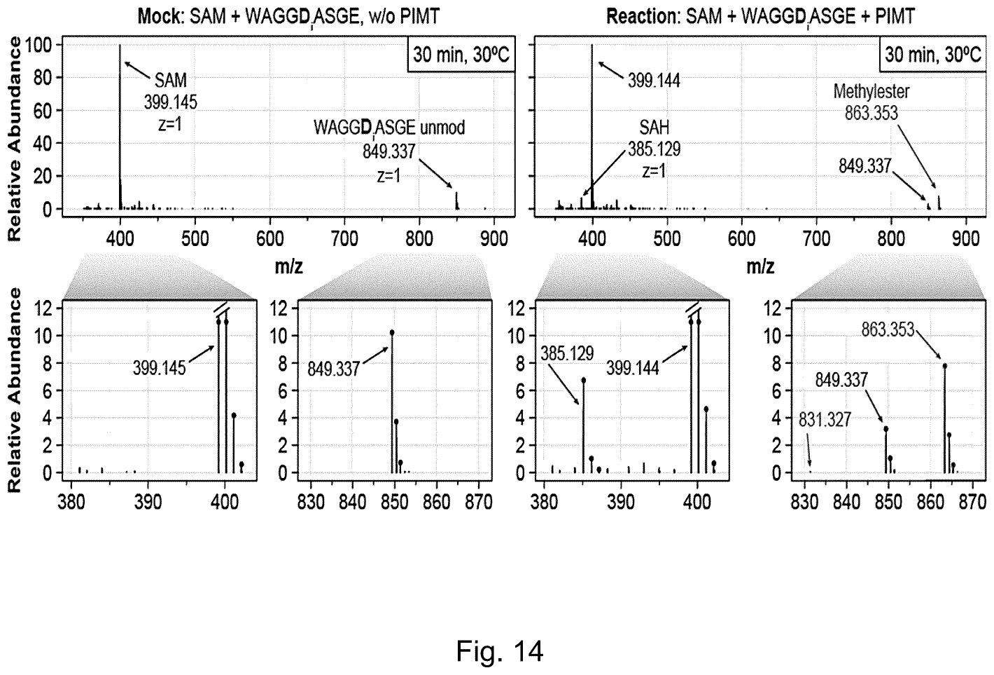

FIG. 14 shows exemplary MS1 spectra for a PIMT reaction with the Isoasp peptide DSIP (WAGGDiASGE—SEQ ID NO: 4) both with and without the addition of SAM. The zoom-in sections focus on SAM and SAH (380-405 m/z) or on DSIP, including the succinimide form, unmodified form, and the methyl ester form (succinimide, 831.327; unmodified, 849.337; methylester, 863.353). The reaction was performed using 25 mM ammonium acetate, 2.1 μM IsoAsp-DSIP in water, 20 μM SAM in 40 mM HCl, 1 μl of PIMT storage buffer (Mock), 1 μl of 530 ng/μl PIMT (Reaction), incubated for 30 min to 2 hrs at room temperature, 30° C., or 37° C., the reaction solution adjusted to 1% FA prior to DI-MS. The reaction was over 50% complete, with the methylester stable under the chosen conditions.

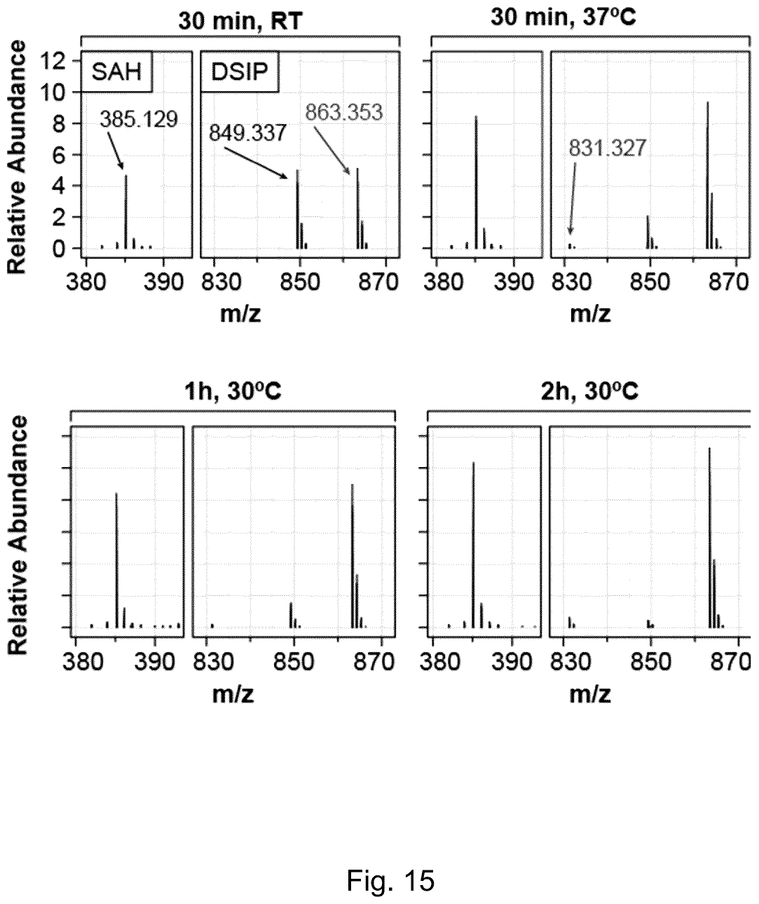

FIG. 15, shows MS1 spectra for reactions performed using for 30 minutes at room temperature, 30 minutes at 37° C., 1 hr, at 30° C., and 2 hrs at 30° C. The 2 hr incubation at 30° C. led to a quasi-complete reaction for IsoAsp-DSIP (SEQ ID NO: 4); however, for IsoAsp PennyK (SEQ ID NO: 6), these reaction conditions proved to be insufficient.

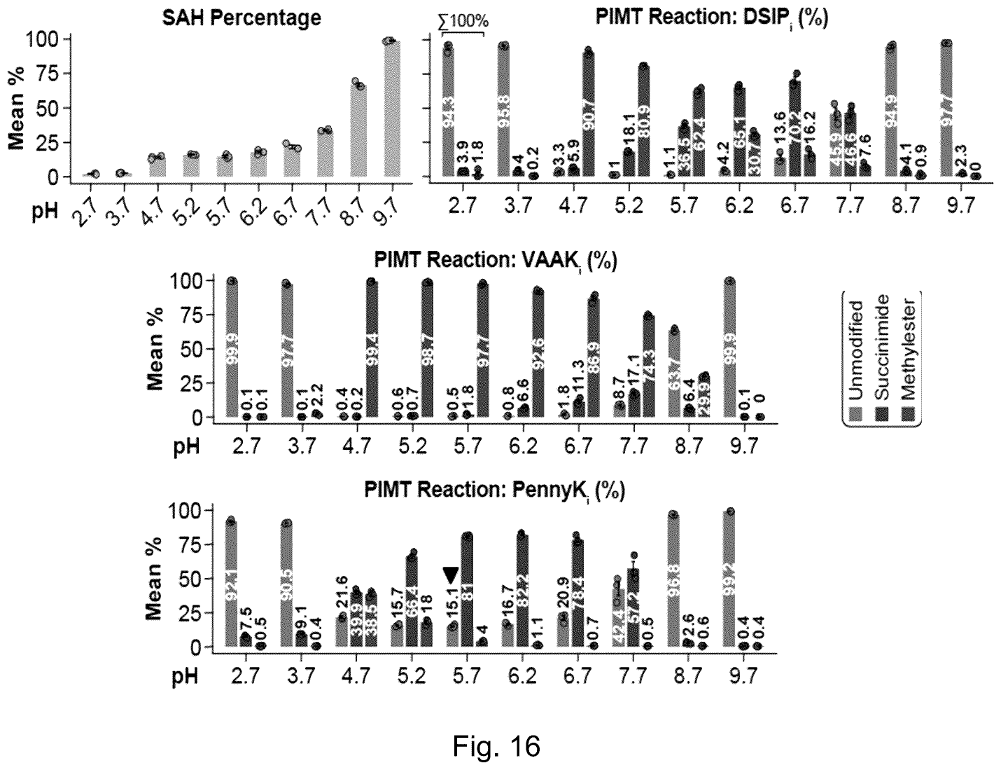

FIG. 16 shows the effect pH has stability of reaction products for PIMT reactions using the DSIP (SEQ ID NO: 4), VAAK (SEQ ID NO: 5), and PennyK (SEQ ID NO: 6) IsoAsp peptides. The peptides were included in the same reaction at a concentration of 1.33 μM each (n=3). The PIMT reactions were performed using the following conditions:

PIMT reaction (100 μl) (μM = pmol/μl)

| | | |
|---|---|---|
| Compound | Concentration | Per reaction |

| NH₄OAc | 10 | mM | n/a | |
|---|---|---|---|---|
| Peptides | Σ4 | μM | Σ400 | pmol |
| SAM | 20 | μM | 2,000 | pmol |
| PIMT | 0.4 | μM | ~1 | μg. |

In the bar graphs, the unmodified peptide form is the left bar, the succinimide form is the middle bar, and the methylester form is the right bar. SAH percentage=(Intensity SAH/(Int SAH+Int SAM))*100, PIMT Reaction % Unmodified=(Intensity Unmodified/(Int Unmod+Int Succ.+Int Methyl.))*100. In these experiments, a pH in the range of 5.2 to 5.7 yielded the best results.

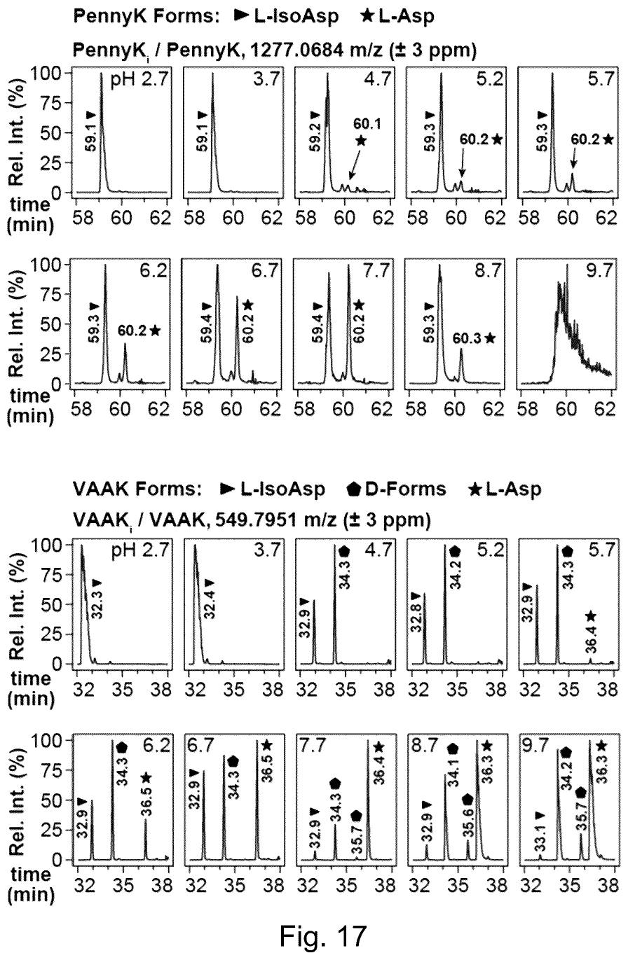

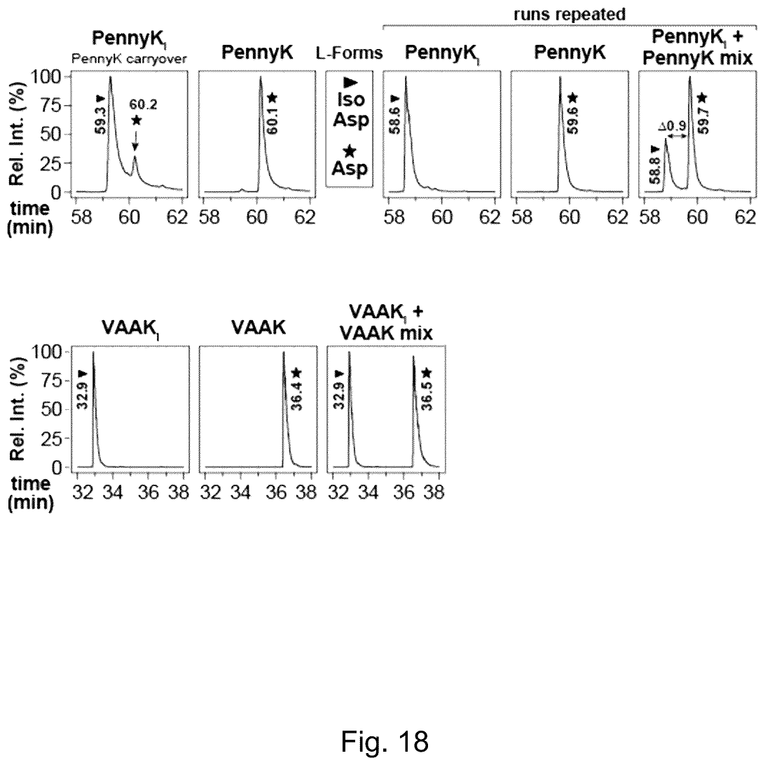

LC-MS was used to determine whether the leftover unmodified peptides following the PIMT reaction were IsoAsp or Asp. At pH ˜5.2 to 5.7, for PennyK, the majority of the unmodified peptide peaks is the IsoAsp form (see FIG. 17). This means that the reaction was still incomplete, but the reaction products can be stabilized at the succinimide level. FIG. 18 shows the retention time for the IsoAsp and Asp isoforms for both PennyK (SEQ ID NOs: 3 and 6) and VAAK peptides (SEQ ID NOs: 4 and 5). IsoAsp versions elute prior to Asp versions of the same peptide.

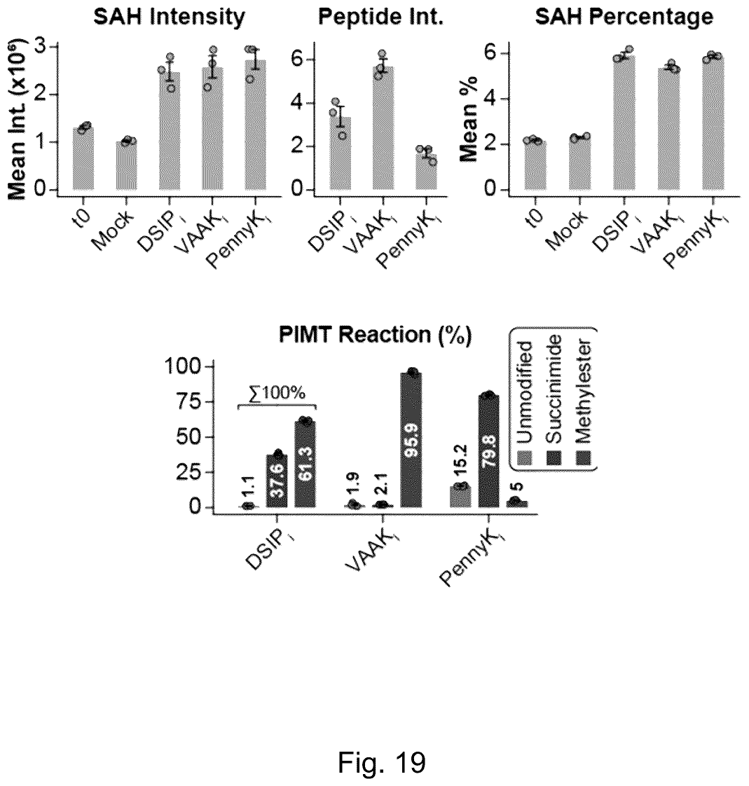

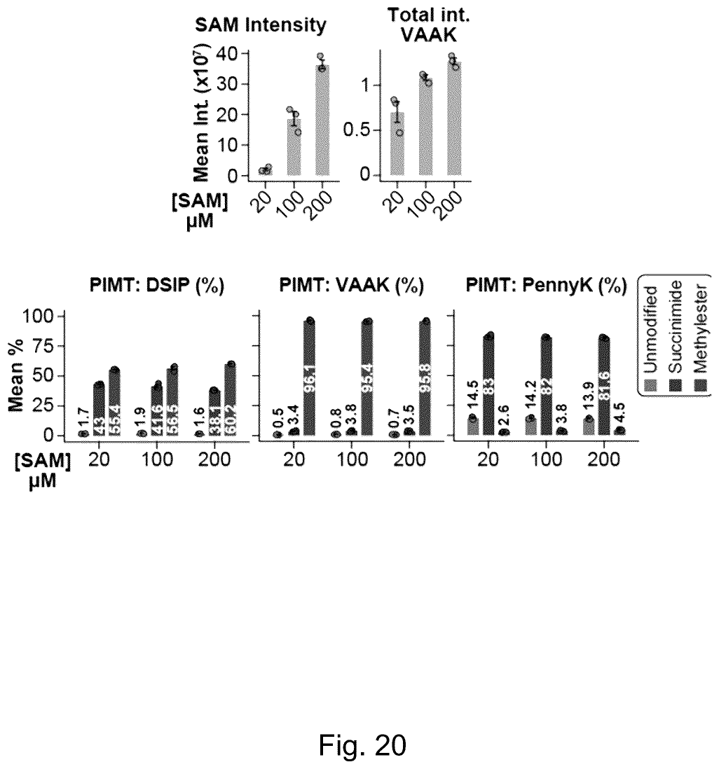

When the IsoAsp peptides were subjected to individual reactions (overnight at 37° C., pH 5.7, 1.33 μM peptide concentration) as opposed to a pooled reaction, the resulting SAH levels were very similar, demonstrating that SAH percentages are a good indicator for overall IsoAsp levels in the sample (see FIG. 19). In a pooled sample, containing IsoAsp DSIP, VAAK and PennyK peptides (SEQ ID Nos: 4, 5 and 6) (3×1.33 μM-Σ4 μM peptide concentration), different input amounts of SAM were sued to determine the impact of SAM concentration on PIMT efficiency (see FIG. 20). Although higher SAM amounts only marginally increased reaction efficiencies for PennyK, no detrimental effects were observed. Since PIMT reaction is compatible with higher SAM input amounts, this allows for higher IsoAsp input amounts as well.

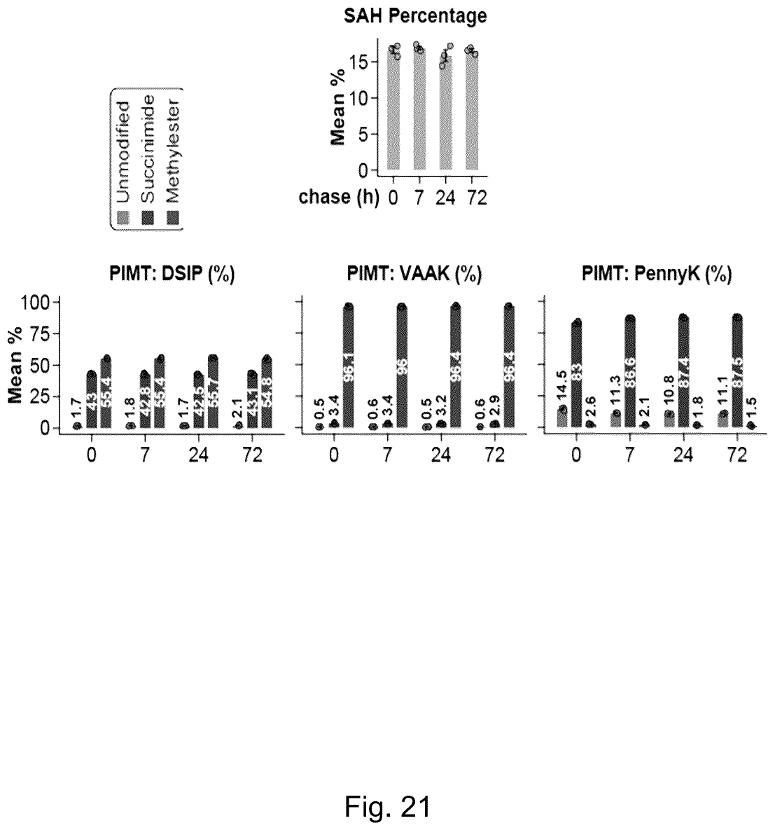

In order to make sure that the PIMT reaction products were stable, a PIMT reaction was adjusted to 0.5% FA, followed by incubation for indicated amounts of time at 8° C. prior to DI-MS analysis. The reaction products (the succinimide form and methylester form) were stable once sample is acidified (see FIG. 21).

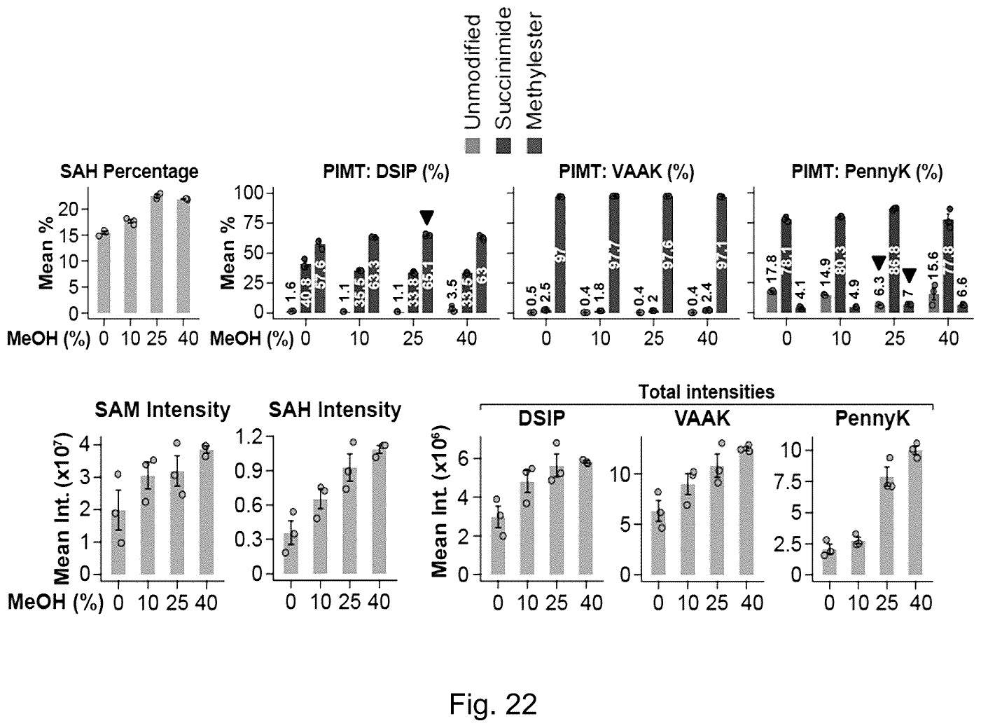

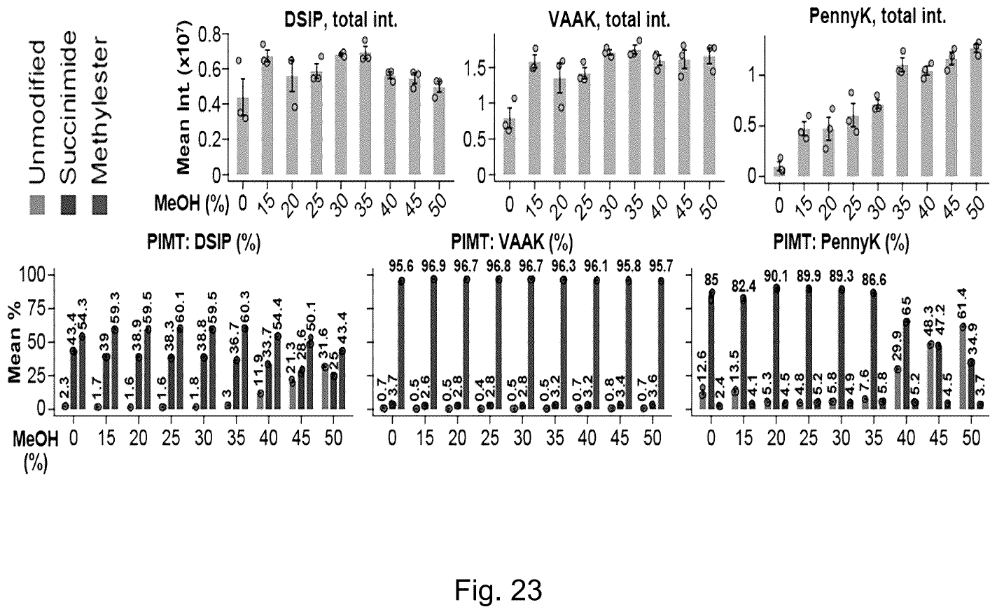

Methanol is part of the IsoAsp reaction cycle. It was suggested that adding MeOH to the PIMT reaction could stabilize some of the reaction products and generally push the reaction equilibrium more into the direction of the reaction products. In reactions (overnight at 37° C., pH 5.7, SAM 20 μM, 3×1.33 μM-Σ4 μM peptide concentration), MeOH improves reaction efficiency for PennyK and in general, while also increasing signal intensity by either improving peptide solubility or by improving peptide ionization efficiency (see FIG. 22). As a follow-up experiment to the initial MeOH sweep experiment, a further experiment was performed to determine the MeOH concentration that supports reaction efficiency best, while also maintaining PIMT enzymatic efficiency. As seen in FIG. 23, final MeOH concentrations from 20% to 30% provided the best results.

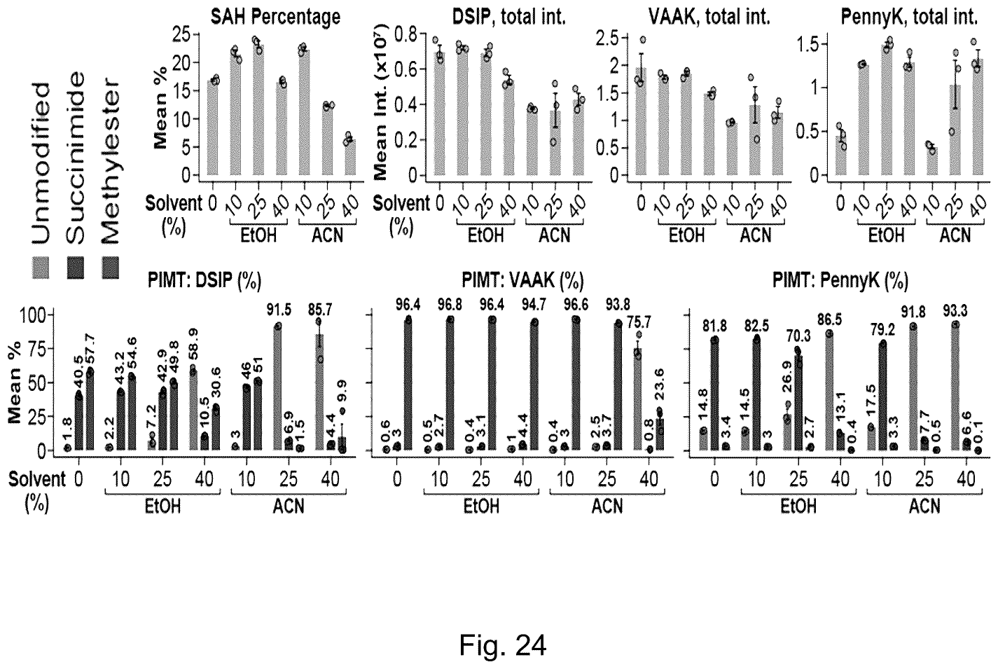

To show that the improvement of PIMT efficiency by adding MeOH was a specific effect and not just a general effect caused by the addition of solvents, the impact of ethanol and acetonitrile on reaction efficiency was also tested. As seen in FIG. 24, PIMT efficiency increase was not seen for ethanol and acetonitrile, indicating that the PIMT efficiency increase is specific to MeOH and not a general solvent-associated effect.

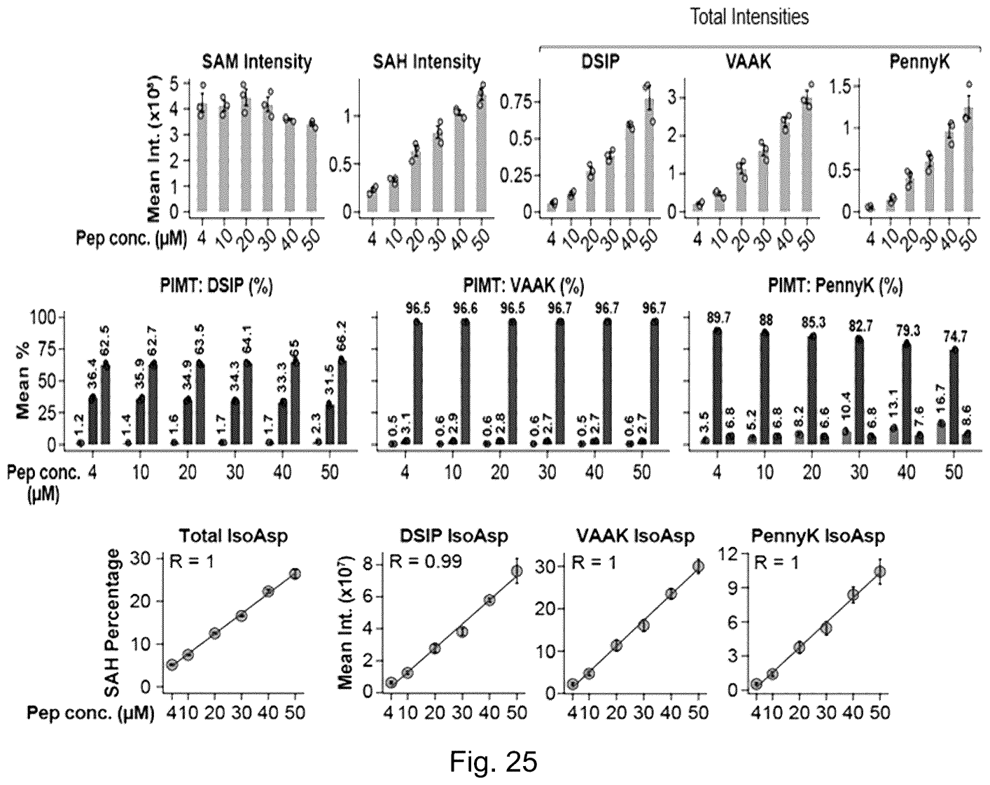

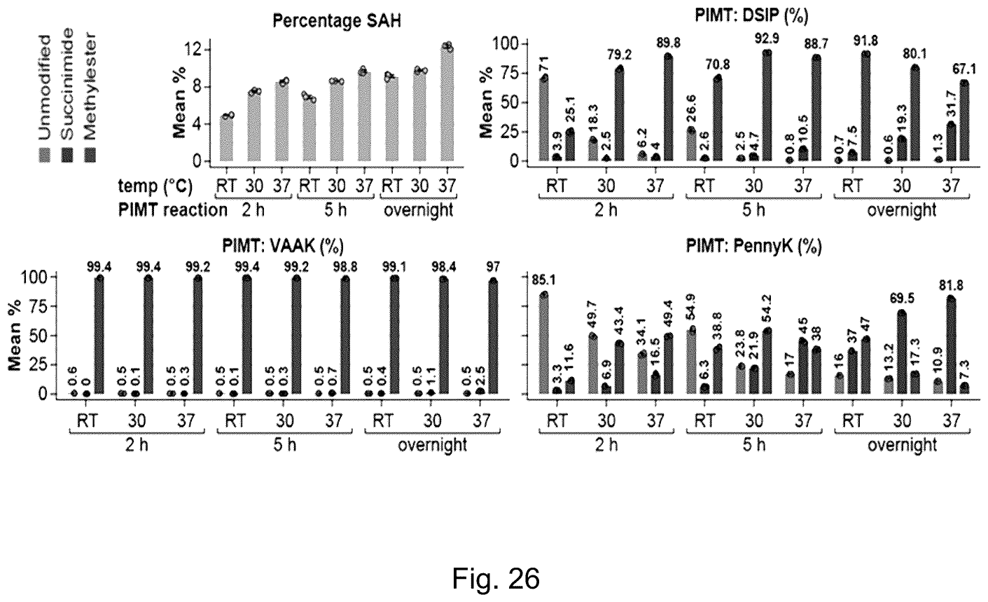

Using the PIMT reaction conditions optimized above, reaction completeness was assessed and correlation across samples when using different peptide quantities as reaction input. Reaction efficiencies for IsoAsp DSIP (SEQ ID NO: 4) and VAAK (SEQ ID NO: 5), even at concentrations of 50 μM (16.66 μM per peptide) were excellent (see FIG. 25). For IsoAsp-PennyK (SEQ ID NO: 6), some residual amounts remained which increase with increasing IsoAsp concentrations, but overall efficiencies for IsoAsp-PennyK were also very good. Correlations across concentrations are excellent both globally across peptides as well as for each peptide individually. To obtain near-complete reaction efficiencies for IsoAsp-PennyK, overnight reactions performed either at 30° C. or at 37° C. are recommended (see FIG. 26).

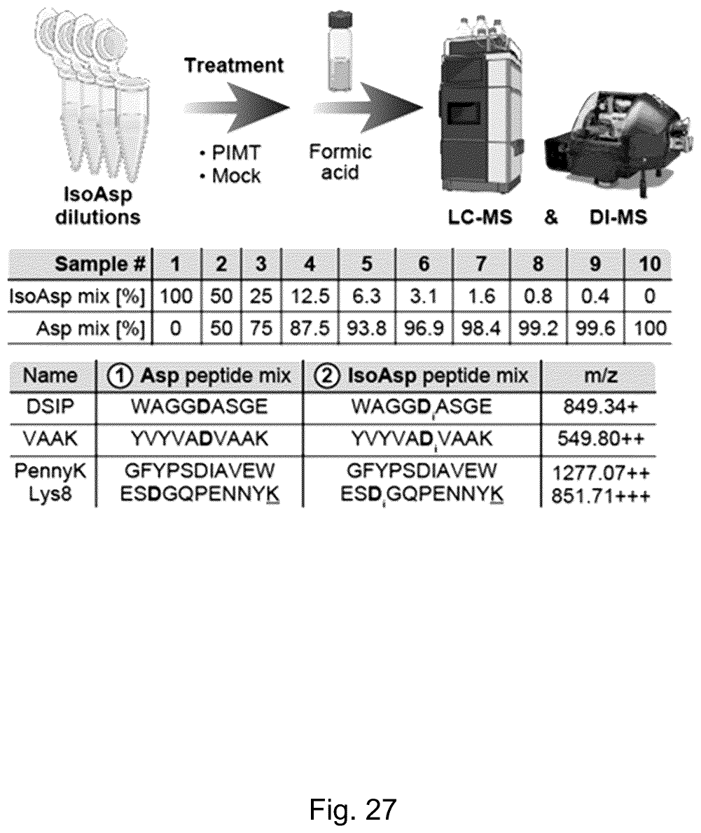

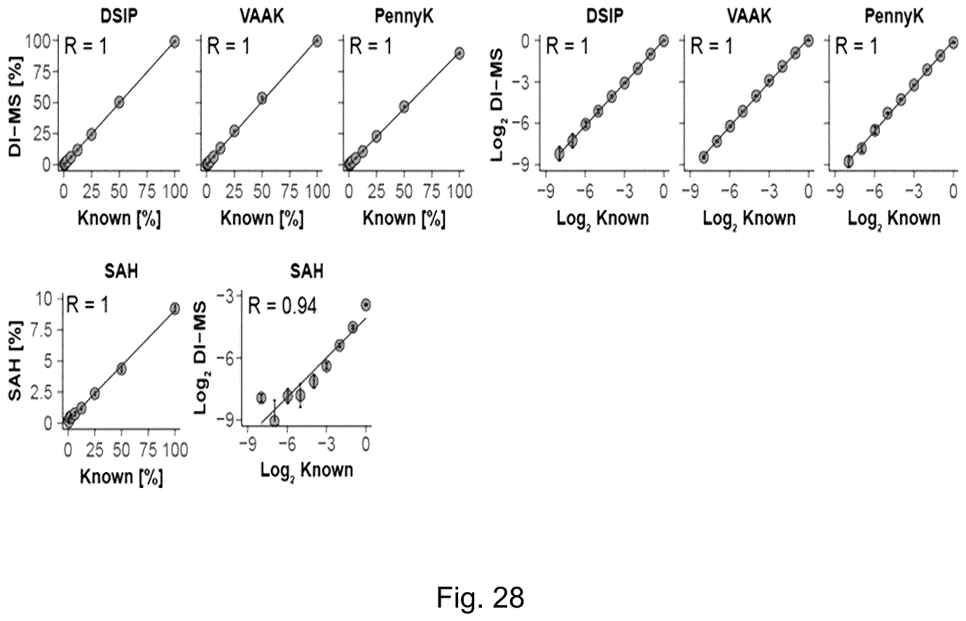

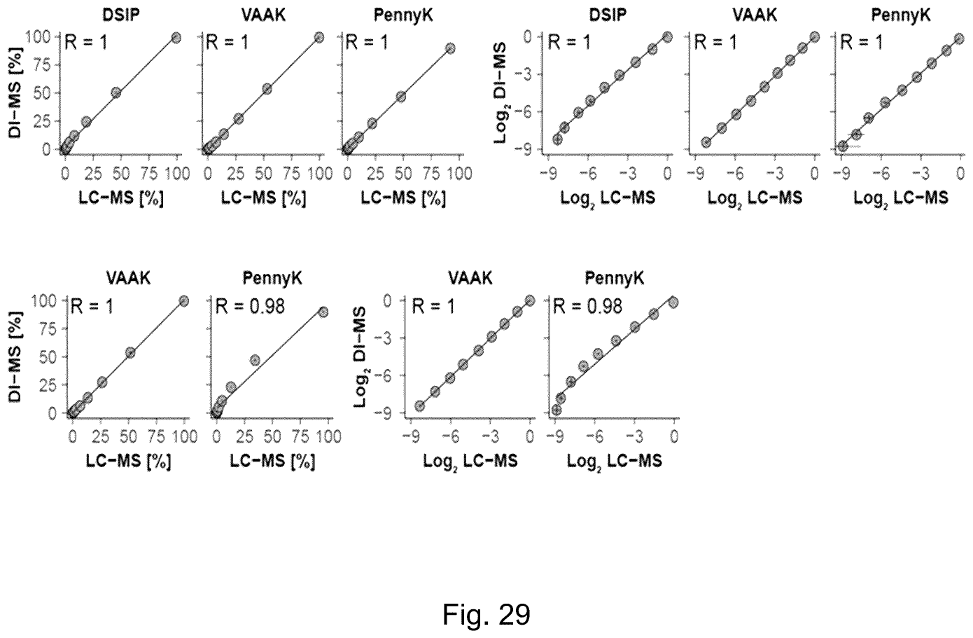

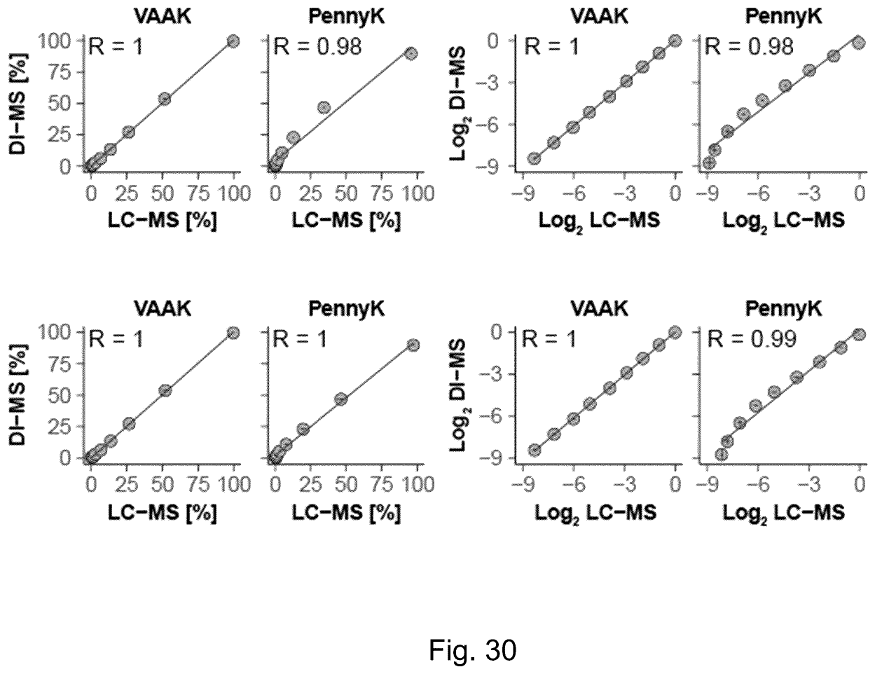

An additional experimental layout was performed for an IsoAsp dilution experiment, where each IsoAsp input amounts decreases across samples but overall peptide amounts stay constant (see FIG. 27). Samples were either PIMT-treated or Mock-treated (control) and analyzed both via DI-MS and LC-MS to compare accuracy of DI-MS with LC-MS (overnight at 37° C., pH 5.7, SAM 200 μM, 3×6.66 μM-Σ20 μM peptide concentration, 25% MeOH concentration). FIGS. 28-30 show that the correlation between: DI-MS (PIMT-treated) and the known concentrations, the DI-MS (PIMT-treated) and LC-MS (PIMT-treated), and DI-MS (PIMT-treated) and LC-MS (Mock-treated) were excellent.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

1. A method of analyzing a first polypeptide having one or more amino acid residues of isoaspartate, aspartate, asparagine, or any combination thereof, wherein each isoaspartate residue has a first mass; the method comprising the steps of:
a) contacting the first polypeptide with a composition comprising protein L-isoaspartyl methyltransferase (PIMT), wherein the PIMT selectively modifies one or more isoaspartate residues, and generating a modified polypeptide comprising one or more modified isoaspartate residues, wherein each of the one or more modified isoaspartate residues has a different mass than the first mass;
b) analyzing the modified polypeptide using a mass spectrometer device, and obtaining mass spectrometry data on the modified polypeptide, wherein the mass spectrometry data comprises a mass-to-charge ratio of the modified polypeptide and/or one or more fragment ions thereof;
c) characterizing the presence and/or amount of modified isoaspartate residues in the modified polypeptide based on the mass spectrometry data.

2. The method of claim 1, wherein the one or more modified isoaspartate residues comprise one or more O-methyl isoaspartate residues and/or one or more succinimide residues thereof.

3. The method of claim 1, wherein each of the one or more modified isoaspartate residues has a mass that differs from the first mass by a pre-selected amount.

4. The method of claim 1, wherein the characterizing step comprises determining a ratio of modified isoaspartate residues in the modified polypeptide to unmodified aspartate residues in the modified polypeptide or to unmodified aspartate residues in an unmodified polypeptide whose mass does not undergo a change after being contacted with the composition comprising PIMT.

5. The method of claim 1, wherein the characterizing step comprises determining a quantitative amount of modified isoaspartate residues and/or unmodified aspartate residues in the modified polypeptide or in an unmodified polypeptide whose mass does not undergo a change after being contacted with the composition comprising PIMT,
wherein the mass spectrometry data comprises signal intensity at one or more mass-to-charge ranges corresponding to modified isoaspartate residues and/or unmodified aspartate residues in the modified polypeptide, and wherein determining the quantitative amount of modified isoaspartate residues and/or unmodified aspartate residues comprises comparing the signal intensity to a signal intensity of a peptide standard comprising a known amount of isoaspartate residues and/or aspartate residues.

6. The method of claim 1, wherein the modified polypeptide has an amino acid sequence and wherein the characterizing step comprises determining one or more amino acid positions in the amino acid sequence that contain the modified isoaspartate residues,
wherein determining the one or more amino acid positions that contain the modified isoaspartate residues comprises the steps of: generating a first distribution of precursor ions from the modified polypeptide, fragmenting a portion of the precursor ions, thereby generating fragment ions, and measuring mass-to-charge ratios of the fragment ions.

7. The method of claim 6 further comprising the steps of: generating a first distribution of precursor ions from an unmodified polypeptide whose mass does not undergo a change after being contacted with the composition comprising PIMT, fragmenting a portion of the precursor ions from the unmodified polypeptide, thereby generating fragment ions from the unmodified polypeptide, and measuring mass-to-charge ratios of the fragment ions from the unmodified polypeptide.

8. The method of claim 1, wherein the first polypeptide is a therapeutic polypeptide or an antibody.

9. The method of claim 1, wherein the first polypeptide is a portion of a therapeutic polypeptide having or suspected of having one or more aspartate residues and/or asparagine residues modified to be isoaspartate residues.

10. The method of claim 1 further comprising performing liquid chromatography on the modified polypeptide prior to obtaining mass spectrometry data on the modified polypeptide.

11. The method of claim 1 comprising injecting the modified polypeptide into an ion source of the mass spectrometer device without performing a liquid chromatography step.

12. The method of claim 11 further comprising the steps of: denaturing the modified polypeptide, treating the denatured polypeptide with a reducing agent, treating the reduced polypeptide with a first solution causing the reduced polypeptide to precipitate out of the first solution, redissolved the polypeptide precipitate in a second solution, and injecting the redissolved polypeptide into the ion source of the mass spectrometer device.

13. The method of claim 1 further comprising contacting a first sample comprising the first polypeptide with a pre-selected amount of S-adenosylmethionine (SAM) concurrently with or prior to contacting the first sample with the PIMT composition, wherein the PIMT composition converts SAM to S-adenosylhomocysteine (SAH); and obtaining mass spectrometry data on the first sample by measuring mass spectrometry signal intensity at a mass-to-charge range corresponding to SAH, thereby obtaining a first SAH signal intensity, and comparing the first SAH signal intensity to a second SAH signal intensity from a calibration sample comprising a known amount of SAH.

14. The method of claim 1 further comprising addition of one or more peptide standards to the first polypeptide, wherein each of the one or more peptide standards has the same amino acid sequence as a polypeptide of interest and has a known amount of isoaspartate residues.

15. A composition for analyzing a first polypeptide having one or more amino acid residues of isoaspartate, aspartate, asparagine, or any combination thereof, said composition comprising:
a) protein L-isoaspartyl methyltransferase (PIMT);
b) one or more peptide standards, wherein each of the one or more peptide standards has a known quantity of isoaspartate residues; and
c) a buffer solution compatible with mass spectrometry analysis.

16. The composition of claim 15 comprising one peptide standard having no isoaspartate residues and one peptide standard having the same amino acid sequence but where all aspartate residues or asparagine residues are replaced with isoaspartate residues.

17. The composition of claim 15, wherein the one or more peptide standards are isotopically labeled.

18. The composition of claim 15 further comprising S-adenosylmethionine (SAM) and a calibration sample containing a standard peptide.

19. The composition of claim 15, wherein the PIMT is a modified PIMT having at least 90% sequence identity to unmodified human PIMT.

20. The composition of claim 15, wherein the PIMT is a modified PIMT having increased resistance to enzymatic digestion, wherein the modification to the PIMT comprises a modification to the N-terminus, C-terminus, and/or peptide backbone, an insertion or substitution of an amino acid with non-canonical side chains, an insertion of one or more amino acids into the PIMT, or a combination thereof.


SEQUENCE LISTING (7 sequences)

SEQ ID NO: 1 - protein, 9 aa, synthetic construct
WAGGDASGE

SEQ ID NO: 2 - protein, 10 aa, synthetic construct
YVYVADVAAK

SEQ ID NO: 3 - protein, 22 aa, synthetic construct
GFYPSDIAVEWESDGQPENNYK

SEQ ID NO: 4 - protein, 9 aa, synthetic construct
  SITE 5: note = isoaspartic acid
WAGGDASGE

SEQ ID NO: 5 - protein, 10 aa, synthetic construct
  SITE 6: note = isoaspartic acid
YVYVADVAAK

SEQ ID NO: 6 - protein, 22 aa, synthetic construct
  SITE 14: note = isoaspartic acid
GFYPSDIAVEWESDGQPENNYK

SEQ ID NO: 7 - protein, 227 aa, Homo sapiens
MAWKSGGASHSELIHNLRKNGIIKTDKVFEVMLATDRSHYAKCNPYMDSPQSIGFQATISAPHMHAYALELLFDQLHEGAKALDVGSGSGILTACFARMVGCTGKVIGIDHIKELVDDSVNNVRKDDPTLLSSGRVQLVVGDGRMGYAEEAPYDAIHVGAAAPVVPQALIDQLKPGGRLILPVGPAGGNQMLEQYDKLQDGSIKMKPLMGVIYVPLTDKEKQWSRWK